(19) United States
(12) Patent Application Publication
(10) Pub. No.: US 2010/0324398 A1
(43) Pub. Date: Dec. 23, 2010

(54) NON-INVASIVE CHARACTERIZATION OF A PHYSIOLOGICAL PARAMETER

(76) Inventor: Jung Tzyy-Ping, Garden Grove, CA (US)

Correspondence Address: BioTechnology Law Group;12707 High Bluff Drive, Suite 200, San Diego, CA, 92130-2037, US

(21) Appl. No.: 12/599,692

(22) PCT Filed: May 12, 2008
(86) PCT No.: PCT/US08/63469

§ 371 Date: Aug. 24, 2010

Related U.S. Application Data
(60) Provisional application No. 60/917,610, filed on May 11, 2007.

(51) Int. Cl. A61B 5/145

(52) U.S. Cl. 600/365

(57) ABSTRACT

The present invention provides a method and device for characterizing a physiological parameter. The method, in one application, uses one or more non-invasive sensors to collect patient data, and may also collect data on environmental conditions. At least some of the patient data has a direct relationship with the physiological parameter, that is, a change in the physiological parameter is reflected in the data set, although the magnitude of the physiological parameter may masked by noise, interference, or other environmental or patient influences. The direct patient data preferably has a generally linear relationship with the physiological parameter, and if not, the patient data is linearized according to an algorithm, table, or other adjustment process. These linearizing processes may be predefined, and may adaptively learn or adjust. A blind signal source process is applied to the linearized data to generate separated signals, and the signal associated with the physiological parameter is identified. The identified signal is scaled or further processed, and the characterization result is presented. Although the method and device are described for use with a human, they may be advantageously used on animals.

### FIELD OF THE INVENTION

Embodiments of the present invention relate to non-invasive devices and methods for characterizing a physiological parameter in a living being, such as a human. In one example, the present invention provides a device and process for estimating a blood analyte concentration level, such as a glucose level.

### DESCRIPTION OF THE RELATED ART

Diabetes is a chronic disease that has no cure. About 20.8 million people (7 percent of the population) of people in the United States were estimated to have diabetes in 2005. As the sixth leading cause of death by disease in 2000, diabetes is costing the U.S. health care system an estimated $132 billion annually. See, National Diabetes Information Clearinghouse, NIH Publication No. 04-3892, November 2003. More serious than the economic costs associated with diabetes is the decrease in the quality of life, serious health complications/consequences, and deaths associated with diabetes.

Diabetes is a group of diseases characterized by high blood glucose levels, which result from defects in insulin production, insulin action, or both. Carbohydrates from food are converted into monosaccharide glucose, which triggers beta cells to release insulin into the blood. Insulin allows for glucose absorption by other cells for energy, molecular conversion or storage. Insulin exhibits control over the conversion of glucose to glycogen for storage in the liver and in muscle cells. However, glucose may be improperly regulated if insulin is produced in insufficient amounts, if insulin is defective or if cells do not properly respond to insulin. This may result in high blood glucose levels, poor protein synthesis and other metabolic derangements.

Hyperglycemia in the diabetic is strongly suspected of being responsible for the long-term effects of diabetes which include cardiovascular disease, arteriosclerosis, blindness, cerebrovascular disease including stroke, hypertension, kidney failure, peripheral vascular disease and premature death. Severe hypoglycemia has similar drastic consequences. In a normal person, the blood glucose level may vary between 60 and 130 milligrams per deciliter, a variance exceeding 100%; whereas, in a diabetic, the levels may vary from time to time from 40 to 500 milligrams per deciliter, a variance of 1150% for hyperglycemia. For hypoglycemia, 60 milligrams per deciliter indicates that treatment is necessary; the glucose may reach a dangerous level of 20 milligrams per deciliter. These large swings of glucose levels must be avoided to prevent the symptoms and complications of the disease. Ideally, the diabetic could conveniently monitor his blood glucose level, and then vary his or her caloric intake, diet and insulin to control the glucose level and thereby avoid the swings. For effective control, frequent blood glucose monitoring is necessary.

Currently, the preferred glucose monitoring technique includes blood sampling. Diabetics prick their epidermis with a needle or lance, usually in the finger, draws a drop of blood, and absorbs the blood on a chemically treated strip of paper. They can then read the glucose level by placing the strip in a glucometer (a spectrophotometer which reads glucose concentrations); or they can compare the color change of the strip with a calibrated color chart. Other methods include measuring the electrical resistance of the strip with a glucometer which is an ohmmeter calibrated in milligrams per deciliter. For effective control, some diabetics must utilize a finger prick four or more times a day.

However, blood extractions for such tests often become a real burden to the diabetic, so they fail to regularly monitor their glucose levels. Diabetic patients may be less likely to routinely monitor their glucose levels due to the invasiveness of the procedure, as well as due to the pain associated with continually pricking their finger. In addition, the chemical reagents used in the tests are quite expensive, particularly in view of the large number of tests required. Accordingly, diabetics may fail to adequately monitor their glucose levels.

Numerous less burdensome or less invasive approaches have been attempted to monitor levels of analytes such as glucose within the body. To date, none have been successful. For example, these approaches have proven not to be accurate enough, or have been so sensitive to environmental conditions that readings are not meaningful. In addition, those devices that produce reasonable data in human subjects typically require substantial calibration data, often involving multiple calibrations (e.g., >20 blood glucose values) over several days. Limitations aside, non-invasive monitoring of physiological parameters, such as non-invasive glucose monitoring, remains the “holy grail” of diabetes management as well as cardiovascular diseases and other conditions that can be monitored or detected using one or more physiological parameters. For example, optical techniques to monitor physiological parameters such as blood analytes are truly noninvasive. The tissue is irradiated, the absorbed or scattered radiation is analyzed, and the information is processed, to provide a measure proportional to the concentration of the blood analyte in the dermal tissue. These techniques include near to far infrared and Raman spectroscopy, polarimetry, light scattering or absorption, and photoacoustic spectroscopy.

One non-invasive approach that has received much attention involves dielectric measurements, such as tissue (skin) electrical impedance measurements. In this approach, the complex impedance is measured over a broad (Hz to MHz to GHz to THz) frequency range. Impedance spectroscopy measures changes in the dielectric properties of the tissue induced by analyte variation. At lower frequencies the response is believed to result from ion rotation in water. This rotation can be affected by both electrolyte (such as NaCl) concentration and substances which alter the solvent viscosity (such as glucose or changes in tissue hydration). At higher frequencies the response is primarily attributed to changes in the dipole moment of the electrolyte constituents. However, the low specificity of pure impedance measurements makes this approach unlikely to succeed. To overcome these difficulties, data are usually obtained over a broad range of frequencies (so-called impedance spectroscopy) and often analyzed by complex statistical algorithms including partial least squares, principal component analysis and neural net analysis.

Non-invasive measurement of skin impedance is described in the literature, for example, in U.S. Pat. Nos. 5,890,489 and 6,517,482; and international patent application No. PCT/US 98/02037 to determine the level of a subject's blood glucose. Impedance based technology has been used for medical purposes since the early 1920's, but it was not until the last few decades that new instruments and methods have become available for various clinical applications—e.g. cardiopulmonary tomography (Metherall et al., Nature 1996; 380: 509-512), skin and tissue hydration (see, e.g., PCT App. No. WO 05/018432 and WO 06/029034, Tagami et al., Invest Dermatol 1980; 75:500-507), detection of dental decay (Longbottom et al., Nat Med 1996, 2:235-237), or of neoplasia (Brown et al., Lancet 2000, 355-892-895; Åberg et al., IEEE Trans Biomed Eng 2004, 51:2097-2102; Åberg et al., Skin Res Technol, 2005, 11:281-286; Emtestam et al., Skin Res Technol 2007, 13:73-78; Hope & Iles, Breast Cancer Res, 2004: 6(2) 69-74) and various types of pathological findings in the skin (see., e.g., Emtestam & Nyrén, Am J Contact Dermatitis 1997, 8:202-206; Hagstromer et al., Skin Pharmacol Appl Skin Physiol 2001, 14:27-33; Nicander et al., Br J Dermatol 1996, 134:221-228; Emtestam et al., Dermatology 1998, 197:313-316, Nicander et al., Skin Res Technol 1997, 3:121-12510), each incorporated herein by reference. Investigations of the dielectric properties of analytes using electromagnetic waves allow one to obtain valuable information on the real-time detection and control of blood analytes. These investigations are also of interest for other applications (see also, e.g., provisional U.S. Pat. App. No. 2006/0025664, Siegel, P. H., IEEE Trans. Microwave Theory and Techniques, 2004; 52(10): 2438-2447; Huo et. al., IEEE Trans. Biomed. Eng. 2004; 51(7): 1089-1094 for RF signals in the micrometer-wave, millimeter/terahertz range).

Therefore, there is a need for a non-invasive but reliable method and apparatus for measuring or characterizing a physiological parameter, such as the concentration of analyte (e.g., glucose) in the body of a mammal.

### SUMMARY OF THE INVENTION

The present invention provides a method and device for characterizing a physiological parameter. The method, in one application, uses one or more non-invasive sensors to collect patient data, and may also collect data on environmental conditions. At least some of the patient data has a direct relationship with the physiological parameter, that is, a change in the physiological parameter is reflected in the data set, although the magnitude of the physiological parameter may masked by noise, interference, or other environmental or patient influences. The direct patient data preferably has a generally linear relationship with the physiological parameter, and if not, the patient data is linearized according to an algorithm, table, or other adjustment process. These linearizing processes may be predefined, and may adaptively learn or adjust. A blind signal source process is applied to the linearized data to generate separated signals, and the signal associated with the physiological parameter is identified. The identified signal is scaled or further processed, and the characterization result is presented. Although the method and device are described for use with a human, they may be advantageously used on animals.

In one example, the present invention provides a glucose monitoring device and method. The glucose monitor non-invasively collects a first set of data that has a direct relationship with the glucose level. The first set of data may be, for example, RF impedance data or infrared data, although many other types of data may be used. The glucose monitor also collects some other data from the patient or from the environment, and uses that data to more effectively process the first set of data. The other data may be, for example, skin temperature, skin humidity, pressure between the non-invasive sensor and the skin, or room temperature. The first set of data may be processed to reduce noise, for example, by processing though a band pass filter, and then linearized according to a predefined algorithm or table. In some examples the linearization process may learn or be otherwise adaptive. The linearized data is passed to an independent component analysis process, where the glucose signal is identified. The glucose signal is then scaled, for example, according to the other data, and presented to the patient as the current glucose level. In one example, the glucose device is a portable and battery powered device. In another example, the glucose monitor is an instrument for use in an office or hospital setting.

Advantageously, the new characterization method and device are relatively insensitive to fluctuating patient and environment conditions. This enables the method and process to more accurately characterize a physiological parameter, and to allow robust characterization in a much wider range of applications. In some applications, the method and device enable fully non-invasive measurements, allowing patients to avoid pain and dread. For example, a glucose monitor using this method is fully non-invasive, avoiding the pain of the needle prick and the mess of the resulting blood. And since the glucose monitor is relatively insensitive to patient or environmental conditions, the diabetic may confidently use the glucose monitor in a wide range of environments. For example, the glucose monitor may provide a good reading irrespective of whether the patient is cold, warm, resting, active, in a warm room, in a cold room, in a place with high humidity, in a dry place, measuring in the morning, or measuring later in the day.

### BRIEF DESCRIPTION OF THE DRAWINGS

FIG. 1 is a flowchart illustrating a process of characterizing a target physiological parameter in accordance with the present invention.

FIG. 2 is a flowchart illustrating a process of characterizing a target physiological parameter in accordance with the present invention.

FIG. 3 is a flowchart illustrating a process of estimating blood analyte concentration levels in accordance with the present invention.

FIG. 4 is a block diagram illustrating components of a device for estimating blood analyte concentration levels in accordance with the present invention.

FIG. 5 is a block diagram illustrating sub-components of the calculation component of FIG. 4.

FIG. 6 is a flowchart illustrating a process of characterizing glucose levels in a human in accordance with the present invention.

FIGS. 7, 8, and 9 are graphs illustrating data and results from an example using a process of characterizing glucose in accordance with the present invention.

FIG. 10 is a flowchart illustrating a process of characterizing a target physiological parameter in accordance with the present invention.

### DETAILED DESCRIPTION OF THE PREFERRED EMBODIMENT

The following detailed description is directed to certain specific embodiments of the invention. However, the invention can be embodied in a multitude of different ways as defined and covered by the claims. In this description, reference is made to the drawings wherein like parts are designated with like numerals throughout.

Generally, the disclosed embodiments describe a process, and an associated device, that is capable of characterizing a target physiological parameter using non-invasive data. In most examples, the process and device are able to characterize the parameter using only non-invasive data, although other examples are discussed. Also, it will be understood that more than one target parameter may be selected, and that the selection of the target parameter may be static or adaptive, and may be manually or automatically chosen. In some embodiments, the present invention relates to a method or device for non-invasively estimating the concentration level of a blood analyte. A plurality of variables may first be non-invasively measured, which may comprise at least one variable that depends on the blood analyte concentration level and/or at least one variable that does not depend on the blood analyte concentration level. The plurality of variables may be nonlinearly transformed and this transformed data may undergo a source separation method. In some embodiments, the blood analyte is glucose.

Definitions

The terms “physiological parameter” and “parameter” are applied to indicate any physiology related value or quantity or data that may be monitored to determine one or more quantitative physiological level and/or activities associated with an individual or subject. Collectively, a plurality of physiological parameters can be collected in a database, or such other stored or measured collection of parameters, over a time interval or period, or at one time point, or continuously to indicate a physiological state of a given subject. For example, physiological parameters that can be measured and indicated include analyte, hydration or moisture, fat or lipose, cardiac output, respiration, oxygen saturation, blood pressure, cellular or tissue characteristics such as cancers, temperature, or other such physiology related information. The general term “parameter” may also refer to an environmental value or data which may directly or indirectly affect or influence a physiological parameter such as ambient information (e.g., room temperature or moisture). The term “property” may be generically interchanged with the term “parameter”.

The term “analyte” refers to a substance or chemical constituent in a biological fluid (e.g., blood or urine) that can be analyzed. In some embodiments, the analyte for measurement by the devices and methods of the present invention is glucose. In other embodiments, the analyte is one or more of lactate, pyruvate, glutamate, oxalate, D-aspartate, L-amino acid, D-amino acid, galactose, sarcosine, urate, ethanol, lysine, cholesterol, glycerol, pyruvate, choline, ascorbate, monoamine oxidases, triglycerides, and uric acid, or other electrolytes.

The term “blood-glucose condition” refers to a condition in which it is desirable to modulate a patient's glucose levels. In some embodiments, blood-glucose conditions include conditions in which it is desirable to reduce blood-glucose levels. For example, high blood-glucose levels can be a blood-glucose condition. In other embodiments, blood-glucose conditions include conditions in which it is desirable to maintain blood-glucose levels at a specific value or within a range of values. In still other embodiments, blood-glucose conditions include conditions in which it is desirable to increase blood-glucose levels. In some embodiments, methods and compositions described herein can be used to first reduce blood-glucose levels and to then maintain the blood-glucose levels at a specific value or within a range of values. Blood-glucose conditions include conditions in which a patient is at risk of developing a blood-glucose condition. In one embodiment, insulin resistance is a blood-glucose condition. In another embodiment, diabetes is a blood-glucose condition.

Impaired glucose homeostasis (or metabolism) refers to a condition in which blood sugar levels are higher than normal but not high enough to be classified as diabetes. There are two categories that are considered risk factors for future diabetes and cardiovascular disease. Impaired glucose tolerance (IGT) occurs when the glucose levels following a 2-hour oral glucose tolerance test are between 140 and 199 mg/dl. IGT is a major risk factor for Type 2 diabetes and is present in about 11% of adults, or approximately 20 million Americans. About 40-45% of persons age 65 years or older have either Type 2 diabetes or IGT. Impaired fasting glucose (IFG) occurs when the glucose levels following an 8-hour fasting plasma glucose test are between 110 and 126 mg/dl.

The term “insulin” refers to a polypeptide hormone (molecular weight of approximately 5700) naturally produced by the pancreas (secreted by beta cells in the islets of Langerhans) of a mammal that controls the amounts of glucose present in the blood by stimulating the uptake of glucose by muscle and adipose tissue. Insulin can exist in various states, such as preproinsulin and proinsulin. The term “insulin” also refers to synthetic versions, such as Humulin® (available commercially from Eli Lilly).

The term “insulin resistance” refers to a condition or disorder in which the tissues of the body fail to respond normally to insulin. Insulin resistance manifests itself in pathologically elevated endogenous insulin and glucose levels and predisposes a mammal to the development of a cluster of abnormalities, including some degree of impaired glucose tolerance, an increase in plasma triglycerides and low density lipoprotein cholesterol (LDL) levels, a decrease in high-density lipoprotein cholesterol (HDL) levels, high blood pressure, hyperuricemia, a decrease in plasma fibrinolytic activity, an increase in cardiovascular disease and atherosclerosis (Reaven, G. M. Physiol Rev. 75(3): 473-86, 1995). Decompensated insulin resistance is widely believed to be an underlying cause of non-insulin dependent diabetes mellitus (NIDDM). Hyperinsulinemia refers to the overproduction of insulin by pancreatic cells. Often, hyperinsulinemia occurs as a result of insulin resistance, which is a condition defined by cellular resistance to the action of insulin. Insulin resistance, as defined above, is a state/disorder in which a normal amount of insulin produces a subnormal biologic (metabolic) response. In insulin-treated patients with diabetes, insulin resistance is considered to be present whenever the therapeutic dose of insulin exceeds the secretory rate of insulin in normal person.

The term “non-invasive” means not requiring breaking the integrity of the body surface. Non-invasive blood analyte concentration level estimation techniques do not require, for example, breaking of the skin to collect blood for analysis, i.e., penetrating the dermis. It may be desirable and still non-invasive, however, to penetrate the outermost layer of the skin, the epidermis, particularly the stratum corneum.

Non-Invasive Characterization of Physiological Parameters

As will be further described in this application, target physiological parameter(s) may be characterized by using data collected using non-invasive measurements of two or more patient or environmental conditions. The measurements may include data collected from each of multiple disparate physical properties, or data collected from multiple disparate measurements of a single physical property. Such target parameters include monitoring or measuring of blood analyte information (e.g., blood chemistry such as oxygen saturation, hemoglobin, glucose and lactate concentrations), body composition (such as lipid or fat composition/content), cellular/tissue characterization or physiological changes, hydration and fluid volumes, body mass or body water content, blood flow or pressure, pulse information, or cardiovascular information. Such information may be specific or generic, and may be collected or stored as further described herein.

In some cases, the disclosed characterization process takes advantage of the fact that improved results sometimes can be obtained by deriving the target physiological parameter from measuring the aggregate effect of changes in or on that parameter. The target physiological parameter can be derived from multiple disparate measurements of physiological or environmental properties. Disparate in this context means that the properties are physically different in nature. The measurements can be from the same parameter or from different parameters. For example, separate measurements can be made of the same parameter over different times or conditions or modalities, or separate measurements can be made of multiple parameters. Alternatively, the measurements can be combinations thereof. The physical properties should each be independently capable of measurement, and preferably have an identifiable relationship, which may or may not be initially obvious, with the character of the target physiological parameter. In this way, a final result may be predicted from the aggregate effect of changes in the physical properties.

For example, target parameter may be derived from measurements of that parameter utilizing different methods. One such example is that changes in hydration level simultaneously affect optical and bio-impedance properties of an animal subject. A particular hydration level implies a particular combination of the values for optical and bio-impedance properties. By deriving the hydration level from the aggregate effect on these properties, a more accurate result can be obtained than can be obtained from either of these properties alone or by merely attempting to compensate for inaccuracies introduced into the system, for example, by environmental changes. It will be understood in this application that the term animal refers to both human and non-human animals.

Alternatively, for example, target parameter can be derived from measurements of that parameter under different conditions or times. For example, a depth selective skin impedance spectrometer is ideal to measure impedance across different areas or layers of the skin. Electrical impedance of biological tissues varies with different settings and frequency. Different settings such as varying energies or frequency intervals contain different types of information. For example, impedance at lower frequencies is influenced by the extra-cellular environment and impedance at higher frequencies by the structure and shape of the cells and the cell membranes (Foster & Schwan, Crit. Rev. Biomed. Eng. (1989) 17:25-104.

Alternatively, for example, it may be desirable to combine measurements of several independent parameters to achieve a high-specificity composite measurement. For example, the method of utilizing bio-impedance information is not specific to any blood analyte and is dependent upon the presence of other biological molecules and electrolytes. In addition, the impedance values are highly dependent on temperature and the volume of tissue being analyzed. However, the combination of impedance with other glucose-dependent physiological variables such as skin temperature, sweat generation and monitoring of other hydrates, blood flow and other cardiovascular information, perfusion, as well as other physiological values may provide increased specificity of the desired analyte information.

In order to properly and reliably characterize the target parameter, the relationship between the measurable physical property and the target physiological parameter should be defined. This can be achieved, for example, by experimentally taking measurements and utilizing such information to obtain a parameter adjustment from a particular combination of results, or alternatively predicting the effects of changes in the physiological parameter on the properties using a mathematical model of animal physiology (see, e.g., T. Forst, T., et al., Diabetes Tech. & Ther., 2006, 8(1): 94-101). In other examples, an algorithmic function may be defined. In other words, independent sources of information on body parameters may be used at the same time in order to obtain the complementary information on unknown parameters. In one embodiment measurements are taken as an independent source of information.

In a more specific example, the disclosed characterization process provides a method of non-invasively determining a target physiological parameter of a subject. The process detects and generates measurement data representing at least two disparate physical properties of the subject, each of the disparate physical properties having a value that varies in dependence on the target physiological parameter and is independently capable of giving a measurement thereof. The measure data is processed to isolate, identify, and characterize the physiological parameter from the aggregate effect of the target physiological parameter on the physical properties. It will be understood in this context that the measurement data may be generated in any manner that creates electrical signals representing the property that are suitable for further processing. They can, for example, be generated by transducer(s) that actively generate(s) signals) from some physical phenomenon, such as pulse rate. Alternatively, the signals could also originate within the body and be, for example, ECG signals, which are merely detected by a passive pick-up.

More than one measurable component may be extracted from the signals during processing. For example, in the case of a complex bio-impedance the final result may depend on such values as aggregate impedance, aggregate phase, and aggregate maximum rate of change of impedance.

In another aspect, a non-invasive apparatus is provided for determining or characterizing a physiological parameter of a patient. The apparatus or device has at least two sensors for generating and/or detecting measurement signals representing disparate physical properties of the subject, each of the disparate physical properties having a value that varies in dependence on the target physiological parameter and is independently capable of giving a measurement thereof. A processor is configured to isolate, identify, and characterize the physiological parameter from the aggregate effect of the target physiological parameter on the physical properties. The processor may derive the physiological parameter from adaptation data stored in a memory or from a mathematical algorithm or model of the animal (human or non-human) physiology. It will be understood that the sensors may be optical, mechanical, or electrical, digital or analog, or other such modality. In a preferred embodiment, at least one of the sensors provides an RF or bio-impedance signal. Typical target physiological parameters that can be characterized include water, electrolyte, fat, analyte, glucose, hemoglobin, lactic acid, cardiac output, respiration, oxygen saturation, blood pressure, pulse, and the like.

The examples disclosed herein provide a device and method for performing non-invasive, accurate, measurement or characterization of physiological parameters of a living body, by combining seemingly disparate physiological parameters, such as dielectric characteristics (e.g., bio-impedance and/or bio/capacitance information), perfusion information, temperature, hydration information, cardiovascular information and such other such physiological information, each which in itself may not provide specific and selective information, to measure and analyze specific aspects of a patient's physiology, such as cardiac output, blood pressure, body composition (e.g. local and total body water, fat and electrolytes) and blood chemistry such as oxygen saturation, hemoglobin, glucose and lactate concentrations. The use of multiple inputs from disparate sources gives more accurate results than can be obtained from a single source, or a single source that is merely compensated. Further, such devices and processes are more immune to changes in environmental and use conditions, and therefore are useable and practical in a wide range of applications and environments.

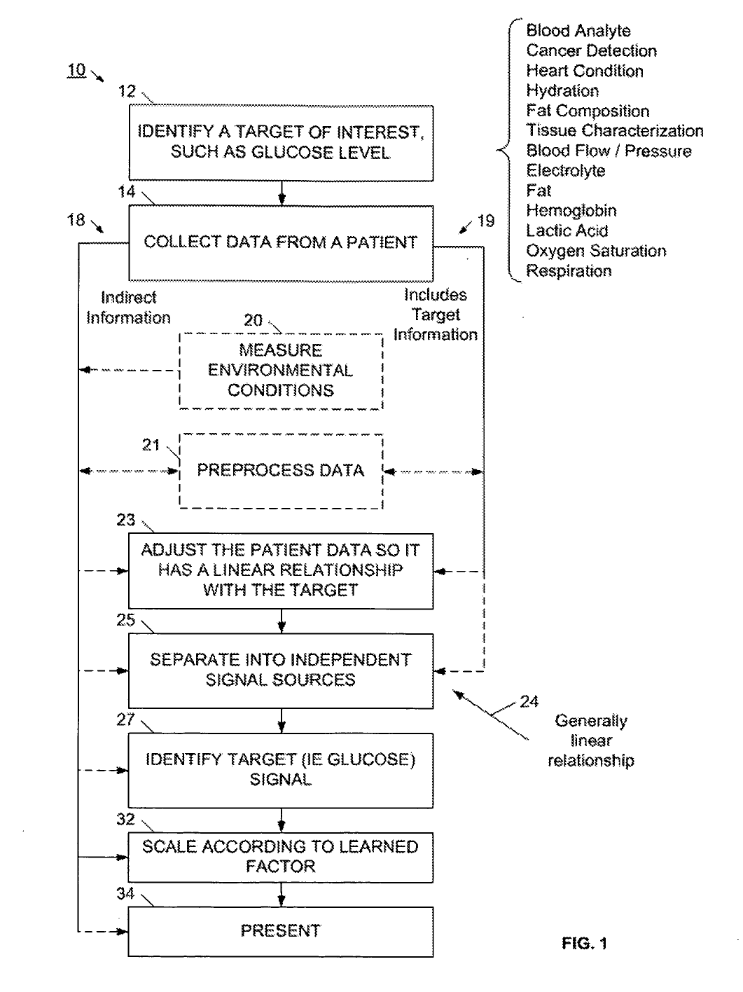

Referring now to FIG. 1, a method of characterizing a physiological parameter is illustrated. Characterization method 10 advantageously enables a simplified and robust process for enabling the characterization of a physiological parameter using non-invasive data. In some example uses, this would enable a simple portable device to noninvasively measure and monitor blood analyte information, such as glucose levels. Such a device would have improved accuracy as well as less sensitivity to environmental conditions. In this way, a patient may easily and painlessly measure and monitor a physiological parameter such as glucose level. With the more patient-friendly processes enabled by method 10, patients are likely to more consistently monitor their physiological parameters, thereby increasing treatment effectiveness and improving an overall quality of life.

Characterization method 10 may characterize more than one physiological parameter, but in many cases will focus on one particular physiological parameter. The physiological parameter may include a blood analyte level, the detection of tissue abnormality, cancer detection, heart rate or heart tissue issue, fat composition, tissue characterization, characterization of blood flow pressure, electrolyte concentration or levels, blood content or hemoglobin levels, lactic acid level, oxygen saturation information, and respiration indicators. It will be appreciated that other physiological parameters may be characterized using method 10. The target physiological parameter is selected for the device as shown in block 12. In one example, the physiological parameter may be a glucose level, although additional or other physiological targets may be selected.

Block 14 shows that data is collected from a patient. Typically, the data is collected for patient using noninvasive sensors. The sensors may be, for example, electrical, RF or other electromagnetic, optical, mechanical, and may be integrated into a single device or may be separated into multiple interconnected devices. In some cases, other patient data may be collected using invasive sensors. For example, some blood information, flow rate information, or blood pressure information may be obtained invasively. Also, it will be understood that some data may be collected and processed in real time, while other data may be collected and stored for later processing. In this way, data may be collected at one time, and then used at another time or other location to provide characterization results. In some cases only a single type of patient data may be collected, and in other processes multiple types of data may be collected. For example, it may be useful to capture RF impedance data for patient, as well as body temperature data. It will be appreciated that sensors for collecting patient data are well known, and will not be described in detail herein.

The data collected from the patient in block 14 generally falls into two categories. First, at least one of the sets of data collected from the patient has a known and direct relationship 19 with the target physiological parameter. This means, for example, that a change in the target physiological parameter causes a change in that set of data. It will be appreciated that the set of data may have other influences that affect the final values of the data set, but these other influences will be eliminated or reduced in other aspects of the characterization process 10. Second, the patient data may also include indirect information 18. Indirect information 18 is not used to directly measure the value for the target physiological parameter, but is used in other aspects of characterization process 10 to provide identification, filtering, or scaling functions. These indirect functions are useful to minimize environmental effects, and to account for the particular current situation of the patient. For example, some physiological parameters may be naturally higher and lower according to the body temperature of the patient. Accordingly, although measuring body temperature does not directly indicate the level of the target physiological parameter, using the body temperature will provide a normalization, calibrating or scaling process to provide more meaningful and consistent information.

Characterization process 10 also allows for the measurement of environmental conditions as shown in block 20. These environmental conditions also provide indirect information useful for providing filtering, identification, or scaling processes according to environmental conditions. For example, some physiological parameters may be naturally higher or lower according to the time of day. By accounting for time of day, characterization process 10 will provide a normalization or scaling process to provide more meaningful and consistent information. It will be appreciated that the environmental conditions may be measured along with the patient data, or may come from other sensors and other devices. The environmental data 20 and the indirect data 18 may be used to drive data processes 21 for preprocessing patient data 19. The preprocessing of methods 21 may be relatively simple filtering processes, or may be configured as more sophisticated adaptive adjustment processes. However, in most cases preprocessing 21 may not be necessary, and if used, will be relatively simple preprocessing methods. For most effective use of characterization process 10, it is desirable that patient data 19 be provided to follow-on process steps with minimal information loss. Accordingly, many of the more complex filtering and processing algorithms, such as PCA, may be undesirable due to their large loss effects.

Through historical information or lab tests, the relationship between the target physiological parameter and the data collected from the patient 19 is understood. In some cases, the relationship may be generally linear, however in many cases the relationship is nonlinear. To increase the effectiveness of follow-on steps in characterization process 10, it is desirable that the patient data 19 have a generally linear relationship to the target physiological parameter. Accordingly, if the patient data is understood to have a generally linear relationship with the target physiological parameter, then the data 19 is passed to the separation process 25. However, if the patient data 19 has a more non-linear relationship, then the patient data 19 is passed to a linearization process 23. In linearization process 23, the patient data 19 is scaled or otherwise adjusted so that the embedded physiological target data has a more linear relationship with the physiological parameter. The linearization process may be implemented in an algorithmic form, as a lookup table, or other modeling or scaling process. Whether the patient data is received directly from block 14, or is first processed in linearization block 23, the data received 24 into the separation block 25 has a generally linear relationship between the data and the target physiological parameter.

In block 25, the linearized data 24 is processed using a blind signal source (BSS) separation process. In one example, the BSS is an independent component analysis (ICA) process. It will be appreciated that other signal separation processes may be used. The BSS process is used to separate the linearized data 24 into separate independent signal sources, with one of the signals directly relating to the target physiological parameter. Independent component analysis (ICA) is a computational method for separating a multivariate signal into additive subcomponents supposing the mutual statistical independence of the non-Gaussian source signals. It is a special case of blind source separation. The statistical method finds the independent components (aka factors, latent variables or sources) by maximizing the statistical independence of the estimated components. ICA can identify linear subspaces of independent components from the signal. In its simplified form, ICA operates an “un-mixing” matrix of weights on the mixed signals, for example multiplying the matrix with the mixed signals, to produce separated signals. The weights are assigned initial values, and then adjusted to maximize joint entropy of the signals in order to minimize information redundancy. This weight-adjusting and entropy-increasing process is repeated until the information redundancy of the signals is reduced to a minimum. More generally, by applying signal separation techniques, linear components can be identified which are independent of each other. Since the invention signal separation techniques can extract original signal from multi-dimensional observation signals mixed with high noise, cleaner signals can be extracted or separated which show higher correlation with the desired physiological parameter. Algorithms for ICA include infomax, FastICA and JADE, but there are many others also.

Once independent signal sources have been identified in block 25, in block 27 the particular target signal is identified. The target signal may be identified due to its particular characteristics or relationship with other signals, or may be identified due to its relationship with other data, such as indirect information 18 or measured environmental conditions. Once the target signal has been identified, it may be scaled to give a consistent normalized result as shown in block 32. Scaling may be assisted with the use of the indirect information or environmental condition information 20. Once the scaled result has been determined, it may be presented as shown in block 34. The result may be indicated on a graphical display, printed, communicated to other devices, or used to set alarms. It will be appreciated that the particular type of presentation may be adjusted according to application needs.

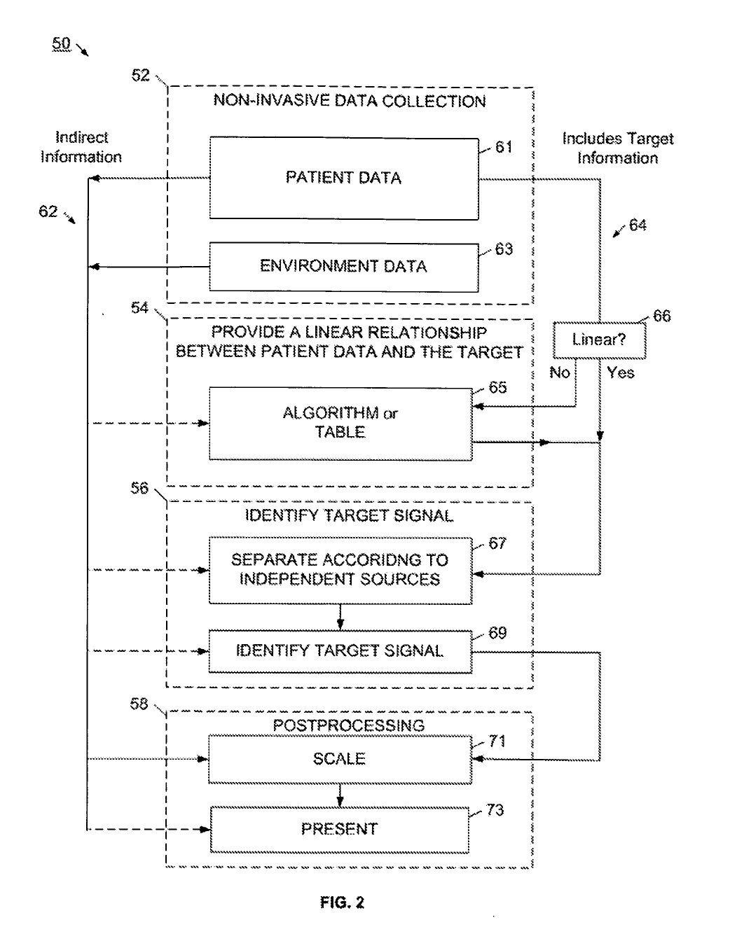

Referring now to FIG. 2, a characterization method 50 is illustrated. It will be appreciated that characterization methods 50 may be used to characterize a wide variety of physiological parameters. It will also be understood that characterization process 50 may be used to characterize a single physiological parameter, or may be used to characterize multiple physiological parameters. Generally, characterization process 50 has four steps: first 52, data is collected from the patient and the environment using non-invasive techniques; second 54, a generally linear relationship is provided between the collected data and the target physiological parameter; third 56, a signal is identified from the data set that is indicative of the target physiological parameter; and fourth 58, the selected signal is scaled in process for presentation. These steps enable characterization process 50 to provide a highly accurate characterization of the target physiological parameter, even under changing patient or environmental conditions. In this way, process 50 may be implemented in a wider range of applications and environments, and may be used with greater confidence and less pain than previous devices or processes.

In step 52, noninvasive data is collected from the patient 61 or from the environment 63. Typically, data is collected from the patient using noninvasive skin-surface sensors. These sensors may be used to measure electrical, optical, temperature, or humidity characteristics, for example. Some of these characteristics may measure surface characteristics, while others may indicate characteristics of underlying tissue or fluids. Some of the sensors may be configured to measure an existing property, such as temperature, while other sensors may actively provide a stimulation. For example, some sensors may provide an RF frequency signal for measuring an RF impedance, while other sensors may provide a light signal for measuring an optical property. It will be understood that a wide range of noninvasive sensors may be used. At least some of the data collected from the patient 61 has information that has a known direct relationship with the target physiological parameter as shown by arrow 264. This means, for example, that a change in the target physiological parameter causes a change in that set of data. Other data from the patient and from the environment may have an indirect relationship as shown by arrow 62. The data 64, which has a known relationship with the target's physiological parameter, preferably has a generally linear relationship prior to use in the identification step 56. Accordingly, in step 66 the target data may be classified according to its linear or nonlinear relationship with the target parameter. In some cases, the determination may be predefined, and in other cases the determination may be made during preprocessing steps. If the data has a linear relationship, then the data is passed to the identification step 56. If the data is not linear, the data is passed through an algorithmic, table, modeling, or other scaling process 65 to adjust the data for a more linear relationship. This linearized data is then passed to identification step 56.

In block 56, the data is first separated into independent sources as shown in block 67, and then the signal associated with the target physiological parameter is identified in block 69. Typically, the separation process will be a blind signal source process, for example, an independent component analysis, or may have another signal separation process applied. With the proper signal identified, the signal is scaled 71, typically using the indirect information 62. The scaled result is then presented as shown in block 73.

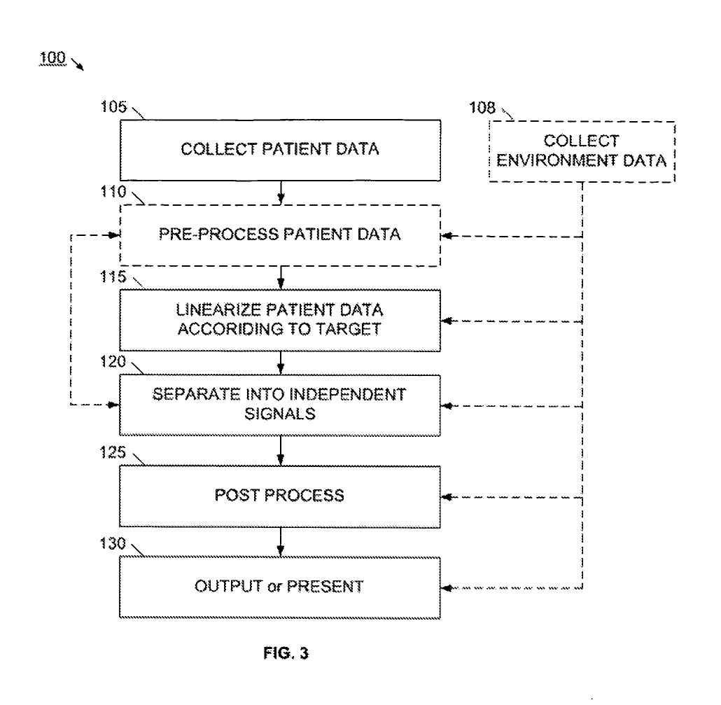

FIG. 3 is a flowchart illustrating a process 100 of estimating a blood analyte concentration level. Depending on the embodiment, additional steps may be added, others removed, and the ordering of the steps rearranged.

Starting at step 105 of process 100, patient data may be obtained. The patient data may be collected using sensors, which may be mechanical, electrical, or optical. The data may be obtained by other input means and/or by measurement means, and the patient data may be obtained from one or more devices. The plurality of properties is preferably non-invasively measured. In preferred embodiments, data representing multiple physical properties are measured nearly simultaneously. In alternate preferred embodiments, the data from each physical property is measured sequentially or over a period of time. In some embodiments, no measurement is invasively made at approximately the same time as the non-invasive measurements are made. In some embodiments, both invasively measured and non-invasively measured properties are measured initially to determine relationship between the collected data and between the blood analyte concentration level, as described in greater detail below. The physical properties may be measured using any signals generated in any manner which represent the property(ies) that are suitable for further processing. They can be generated, for example, by transducer(s) that actively generate and record signals from some physical phenomenon. Alternatively, the signals could also originate within the body and be detected by active or passive pick-up.

In some embodiments, at least one of the measure physical properties depends on a blood analyte concentration level. One or more of the physical properties may be related to a patient. One or more of the physical properties may be measure using a dielectric measurement. The dielectric measurement may be an impedance spectroscopy measure. The impedance measure may be a radio-frequency (RF) or bio-impedance impedance measure. One or more of the physical properties may be measured using an optical sensor and detector (e.g., IR, Doppler, reflectance, Raman, polarization, fluorescence, etc.). One or more of the physical properties may be measured using a capacitance variable. One or more of the physical properties can be measured using audio or pulse wave measurements. One or more of the physical properties may be measured using a current variable. One or more of the physical properties may be measured using an imaging technique. One or more of the physical properties may be measured using a electromagnetic measurement. Other such methods will be known to those skilled in the art. The physical properties may be measured by a device positioned on and/or over the skin of a patient, and in some cases, may include an invasive device, such as an implant. In one embodiment, combinations of such measurements can be made.

In some embodiments, at least one of the types of patient data does not directly depend on a blood analyte concentration level. Without wishing to be bound to any particular theory, patient data that is insensitive to the blood analyte concentration level may still be useful for the estimation, as it may affect another physical property that is sensitive to the concentration level. Such physical properties include temperature (e.g., ambient, skin or internal), moisture such as sweat generation, perfusion or blood flow, internal or external pressure (e.g., blood pressure or device pressure), blood oximetry or pulse, as well as ECG or EEG values. In some embodiments, a patient data that does not directly depend on a blood analyte concentration level is estimated rather than measured. For example, the room temperature may be approximately known, so it may be estimated rather than measured. One or more of the measure data sets may be related to a patient, such as the body temperature of a patient. One or more of the measured data sets may be related to the environment, such as the room or ambient temperature. For example, block 108 shows that environmental data may be collected, such as time of day, humidity, temperature, ambient light, and the like. While in some embodiments, one or more of the measured data sets are related to the time of day, in other embodiments, none of the measured data sets are related to the time of day. In some embodiments, one blood analyte concentration level may globally depend on another blood analyte concentration level but may not be sensitive to day-to-day fluctuations. To illustrate this concept, a diabetic patient may be more likely to have high blood pressure, but blood pressure readings may be uninformative as to the patient's instantaneous glucose concentration levels.

In one embodiment, RF impedance measurement data is collected as patient data. Electrical impedance of biological tissues varies with an applied frequency, current or voltage signal, and the impedance values may be measured and collected. Different frequency intervals contain different types of information. For example, impedance at lower frequencies is influenced by the extra-cellular environment and impedance at higher frequencies by the structure and shape of the cells and the cell membranes. In impedance spectra, this information is diffusely spread and overlapped in the whole frequency. The impedance can also be generated from the exchange of energy from an external power source, e.g., both alternating current and/or voltage and direct current and/or voltage. The alternating current and/or voltage can include alternating or a range of frequencies. A correct frequency needs to be chosen in order to develop a sensor based on impedance spectroscopy that will be sensitive to electrical changes in the blood, tissue or body. For example, glucose changes can be selected within the range of 10 Hz and 50 GHz, preferably between 1 KHz and 100 MHz. Other measurements of changes in tissue characteristics, such as tumor detection, can be selected from as low as 1 Hz to 50 GHz, preferably between 10 Hz and 200 KHz.

The measured data sets may include other physiological data that include but are not limited to one or more of a patient's skin temperature, a patient's body temperature or ambient temperature, a patient's skin moisture, a patient's blood flow or pressure or other vascular activity, a patient's skin/tissue hydration, an ECG variable, an EEG variable, an oxygen saturation variable, air temperature, humidity, atmospheric pressure, a skin-device pressure variable, and a device movement variable.

It is preferred that a plurality of measurements be taken from different and analogous sensors.

Pre-Processing

At step 110 of process 100, one or more data sets are pre-processed. As employed herein, pre-processing comprises preparing the input data (signals or information) for signal separation processing 120. In some embodiments, step 110 is not part of process 100. The one or more data sets may be measured variables and/or input variables. The pre-processing may include a variety of processes, including identifying, categorizing, filtering, transforming, calibrating, resampling, smoothing, transforming, normalizing, selecting, registration, quantization, and other similar processes, individually or in combination, such that relevant information is not lost. Preferably, the data will retain as much information, e.g., raw, to retain as much relevant or potentially relevant information as possible, contrary to steps such as normalizing or averaging, or other processes which remove information. For example, principal components analysis (PCA) is a technique for simplifying a data set by reducing multidimensional data sets to lower dimensions for analysis. Although such steps simplify processing, information which is important or potentially important is permanently lost. Pre-processing may be performed on each data set or an aggregate set of data, preferably such that the number of output components resulting from pre-processing step is equal to the number of input components.

Preferably, the pre-processing step 110 involves identifying and/or categorizing input information to determine whether further pre-processing is required. Information which identifies the input data sets as non-activity or static or null information, duplication, non-linearity, or other such characterization would improve processing. Filtering can include filters on each data signal or on the aggregate of signals, such as removal of non-relevant inputs. Pre-processing may be static or adaptive. For example, the output of the separation signal may influence the pre-processing step as a feed forward or feedback loop, or alternatively, the filter can be designed learned filters from prior knowledge or empirical data acquisition. Pre-processing may also include the combining of two or more measurements. For example, two or more impedance readings may be combined into a single variable if they are identical.

Nonlinear Transformations

At step 115 of process 110, one or data sets are nonlinearly transformed, if and when identified. The nonlinear transformation may be a nonlinear filtering, a look-up table, or an algorithm, for example. In some embodiments, one or more data sets that depend on a blood analyte concentration level are nonlinearly transformed while no data sets that do not depend on the blood analyte concentration level are nonlinearly transformed. For example, a data set derived from measuring an RF impedance may undergo a linearization process, whereas data regarding room temperature may not. In some embodiments, one or more data sets that do not depend on a blood analyte concentration level are nonlinearly transformed while no variables that depend on the blood analyte concentration level are nonlinearly transformed. In some embodiments, both one or more data sets that depend on a blood analyte concentration level and one or more data sets that do not depend on the blood analyte concentration level are nonlinearly transformed.

The nonlinear transformation may be a variety of transformations, including, without limitation, single variate transformation, polynomial transformations, e.g., f(x)=a·x^{b}+c, trigonometric transformations, e.g., f(x)=cosh(x), exponential transformations, e.g., f(x)=1/(1+exp(−x)), logarithmic transformations, e.g., f(x)=log(x), and the like. It will be understood that if nonlinear transformations are applied to multiple variables, different or the same nonlinear transformations may be applied to the variables.

The specific nonlinear transformations may be determined by known relationships between a data set or physical property and another data set or between a data set and a known physiological parameter, such as blood analyte concentration level. For example, a known relationship between an impedance data set and a body temperature data set may be established and one or both of these data sets may be nonlinearly transformed based on this known relationship. In some embodiments, the nonlinear relationship is established using test data. The test data may include a set of non-invasively measured data sets and corresponding invasively measured data sets. In some embodiments, a variety of nonlinear transformations are performed on one or more non-invasively measured data sets and the estimated blood analyte concentration levels are compared to invasively measured data sets. The most accurate transformation may then be used to estimate the blood analyte concentration levels when only the non-invasively measured data sets are measured. In some embodiments, a learning rule is used to estimate a nonlinear transformation based on the test data. The learning rule may be constrained. The learning rule may include a priori constraints and/or derived constraints. The learning rule may comprise a neural network.

In some embodiments, the nonlinear transformation step 115 is performed prior to any signal separation step 120, while in other embodiments a signal separation step 120 precedes the nonlinear transformation step 115. In some embodiments, the nonlinear transformation step 115 is performed after the pre-processing step 105. In some embodiments, the nonlinear transformation is at least partially adaptive.

Source Separation

In some embodiments, a linear mapping, z, is computed from variables, Y, such that the linear mapping, z, is correlated with the desired physiological parameter, such as a blood analyte concentration level.

z=WY Eq. 1

The variables, Y, may comprise non-invasively measured variables that may have been pre-processed, including transforming any Y that is nonlinear. Although the mapping may be substantially insensitive to personal and/or environmental changes, the goal is to have a system that is robust to such changes. Accordingly, the prediction weight, W, may be determined by a variety of methods. For example, test data may be used to establish a linear regression between invasively measured blood analyte concentration levels and the variables Y. However, preferably, a more complex regression model such as a neural network can be used to determine the prediction weight.

At step 120 of process 100, a source separation process is used to separate an independent signal from at least two data sets. In some embodiments, step 120 is not part of process 100. In one example, signal separation process(es) 120 includes signal separation or blind source extraction (BSE) techniques known to those skilled in the art, including non-orthogonal transformation methods. Each input data set is considered a channel of input signals to the transformation. The signal separation method is applied to the channels of input signals to separate a multivariate signal into statistically substantially-independent components. In one specific implementation, a blind source separation (BSS) or an independent component analysis (ICA) or an independent vector analysis (IVA) method is used as the signal separation process. Blind source extraction (BSE) is a techniques that extracts a small subset of source signals from high-dimensional observed signals. See, for example: Cichocki, A., Amari, S., Adaptive Blind Signal and Image Processing: Learning Algorithms and Applications, John Wiley & Sons, New York (2002); Cichocki, A., et al.: A Blind Extraction of Temporally Correlated but Statistically Dependent Acoustic Signals, Proc. of the 2000 IEEE Signal Processing Society Workshop on Neural Networks for Signal Processing X (2000) 455-46; Smith, D., Lukasiak, J., Burnett, I.: Blind Speech Separation Using a Joint Model of Speech Production, IEEE Signal Processing Lett. 12 (11) (2005) 784-787; Zhang, Z.-L., Yi, Z.: Robust Extraction of Specific Signals with Temporal Structure, Neurocomputing 69 (7-9) (2006) 888-893; Barros, A. K., Cichocki, A.: Extraction of Specific Signals with Temporal Structure, Neural Computation 13 (9) (2001) 1995-2003; Cichocki, A., Thawonmas, R.: On-line Algorithm for Blind Signal Extraction of Arbitrarily Distributed, but Temporally Correlated Sources Using Second Order Statistics, Neural Processing Letters 12 (2000) 91-98; Mandic, D. P., Cichocki, A.: An Online Algorithm for Blind Extraction of Sources with Different Dynamical Structures, Proc. of the 4th Int. Conf. on Independent Component Analysis and Blind Signal Separation (ICA 2003) (2003) 645-650; Liu, W., Mandic, D. P., Cichocki, A.: A Class of Novel Blind Source Extraction Algorithms Based on a Linear Predictor, Proc. of ISCAS 2005, pp. 3599-3602; Liu, W., Mandic, D. P., Cichocki, A.: Blind Second-order Source Extraction of Instantaneous Noisy Mixtures, IEEE Trans. Circuits Syst. II 53 (9) (2006) 931-935.

Independent component analysis (ICA) is a computational method for separating a multivariate signal into additive subcomponents supposing the mutual statistical independence of the non-Gaussian source signals. It is a special case of blind source separation. The statistical method finds the independent components (aka factors, latent variables or sources) by maximizing the statistical independence of the estimated components. ICA can identify linear subspaces of independent components from the signal. In its simplified form, ICA operates an “un-mixing” matrix of weights on the mixed signals, for example multiplying the matrix with the mixed signals, to produce separated signals. The weights are assigned initial values, and then adjusted to maximize joint entropy of the signals in order to minimize information redundancy. This weight-adjusting and entropy-increasing process is repeated until the information redundancy of the signals is reduced to a minimum. When applied to signal Y, the ICA method may identify a number of subspaces for which signals are independent of each other. More generally, by applying signal separation techniques, linear components can be identified which are independent of each other. Since the invention signal separation techniques can extract original signal from multi-dimensional observation signals mixed with high noise, cleaner signals can be extracted or separated which show higher correlation with the desired physiological parameter. Algorithms for ICA include infomax, FastICA and JADE, but there are many others also.

Although process 120 may use an ICA process, it will be understood that other signal separation processes may be used in accordance with this disclosure, including extensions of ICA. Many different algorithms for solving the separation can be found in the literature, including some of the better known algorithms such as JADE (Cardoso & Souloumiac (1993) IKE proceedings-F, 140(6); SOBI (Belouchrani et al. (1997) IEEE transactions on signal processing 45(2)); BLISS (Clarke, I. J. (1998) EUSIPCO 1998)); Fast ICA (Hyvarinen & Oja (1997) Neural Computation 9:1483-92); and the like. A summary of the most widely used algorithms and techniques can be found in books and references therein about ICA and BSS (e.g., PCT Application Nos. WO 05/052848 and WO 03/073612; Girolami, M., Advances in Independent Component Analysis, Springer (December 2006); Stone, J. V., Independent Component Analysis: A Tutorial Introduction, MIT Press (September 2004); Roberts and Everson, Independent Component Analysis: Principles and Practice, Cambridge University Press (March 2001); Hyvarinen et al., Independent Component Analysis, 1st edition (Wiley-Interscience, May 2001); Haykin, Simon. Unsupervised Adaptive Filtering, Volume 1: Blind Source Separation. Wiley-Interscience; (Mar. 31, 2000); Haykin, Simon. Unsupervised Adaptive Filtering Volume 2: Blind Deconvolution. Wiley-Interscience (March 2005); and Mark Girolami, Self Organizing Neural Networks: Independent Component Analysis and Blind Source Separation (Perspectives in Neural Computing) (Springer Verlag, September 1999). Singular value decomposition algorithms have been disclosed in Adaptive Filter Theory by Simon Haykin (Third Edition, Prentice-Hall (NJ), (1996).

Also contemplated are extensions of ICA developed to allow ICA applicable to a wider range of data analysis area. These extensions include noisy ICA, independent subspace analysis, multidimensional ICA, (post-)nonlinear ICA, tree-dependent component analysis, subband decomposition ICA, independent vector analysis (IVA, PCT Application No. PCT/US2006/007496; U.S. Provisional App. Nos. 60/891,677, 60/777,900 and 60/777,920; Kim et al., Independent Vector Analysis: An Extension of ICA to Multivariate Components. ICA 2006: 165-172; Lee, et al., Complex FastIVA: A Robust Maximum Likelihood Approach of MICA for Convolutive BSS. ICA 2006: 625-632; Taesu Kim, “Independent Vector Analysis,” Ph. D. Thesis, KAIST, February, 2007; each incorporated herein by reference.

Other non-orthogonal transformation methods contemplated for source separation, such as Varimax, Promax, variational methods and so forth, can also be used. In one experiment, one-lead ECG signals are isolated and time-aligned into 5000 heartbeat cycles, and separated by an ICA method into 150 components. Although process 120 may use an ICA process, it will be understood that other signal separation processes may be used in accordance with this disclosure. The source separation process 120 may be a linear source separation process. In some embodiments, a first data set that depends on a blood analyte concentration level also depends on a second data set. A source separation process as described herein may be used to improve a correlation between the first variable and the blood analyte concentration level. The source separation process may be at least partially adaptive. The source separation process may comprise one or more constraints. The constraints may include a priori constraints and/or derived constraints. Parameters, equations and/or other properties related to the source separation process may be determined by a learning rule, such as a neural network.

Post-Processing

At step 125 of process 100, one or more outputs from the separation process described herein may undergo post-processing. In some embodiments, step 125 is not part of process 100. In some embodiments, one or more signals that have been separated by the source separation process are post-processed. For example, the post processing steps may include identifying the signal source associate with the target physiological parameter. In other cases, the signal separation process may be adjusted to only pass the proper signal to post processing. In some embodiments, one or more variables that are estimated to be correlated to a known physiological parameter, such as a blood analyte concentration level, undergo post-processing.

The post-processing may include scaling and/or applying an offset. The scaling factor and/or the offset may be determined by a test data set, or may be responsive to another patient data set or environmental data set. The test data set may comprise both separated data derived from non-invasively measured variables and invasively measured data. The post-processing may include combining at least two of the separated variables. In some embodiments, the post-processing comprises linear processes. In some embodiments, the post-processing comprises non-linear processes. In some embodiments, the post-processing is at least partially adaptive. In some embodiments, the post-processing comprises constraints, which may include a priori constraints and/or derived constraints.

Output Variables

At step 130 of process 100, one or more characteristics of the target parameter are output. In some embodiments, step 130 is not part of process 100. A system and/or method described herein may estimate and may output a desired physiological parameter; a blood analyte concentration level, for example. In some embodiments, an output characteristic is a single estimated concentration level. In some embodiments, the output characteristic comprises a range of concentration levels, or a rate of change. The range of levels may indicate, for example, the confidence in the calculation. The range of levels may indicate that the level is within a specific physiological range. For example, after the concentration level is calculated, the system and/or method may simply indicate that it is within a range of levels deemed normal.

In some embodiments, the output variable indicates whether the estimated physiological parameter level is above or below a threshold value. For example, an output variable may indicate that a blood analyte concentration level is too high, such that a counteracting drug such as insulin should be injected. In some embodiments, the output characteristic is not a number. The output characteristic may be an interpretation of a concentration level. For example, the output variables may comprise “below acceptable level”, “acceptable level”, and “above acceptable level”. The output characteristic may provide instructions to the patient based on the blood analyte concentration level. The instructions may relate to the type, timing, and/or dosage of treatments to be administered, to dietary advice, and/or advice about seeking professional assistance, such as a doctor or an emergency unit. In some cases, the output may be an alarm. In some embodiments, the output variable comprises a concentration level relative to another concentration level. For example, the output variable may comprise a ratio of the blood analyte concentration level relative to the concentration level most recently measured or relative to the average concentration level deemed to be acceptable. In some embodiments, the output characteristic comprises a scaled version of the blood analyte concentration level. For example, the concentration level may be scaled to a 1-10 scale, such that a “1” indicates concentration levels far below acceptable values and a “10” indicates concentration levels far above acceptable values. In some embodiments, the output may be useful to the patient, to the primary care giver or physician, or the medical provider, including emergency personnel.

Post-processing components of methods and/or systems described herein may comprise conversion components to convert estimated blood analyte concentration levels to an output characteristic described herein. In some embodiments, the conversion depends on details of the concentration level calculations, such as when the output characteristic indicates the confidence in the estimate. In some embodiments, the conversion is a fixed conversion, such as implementing a fixed relationship between blood analyte concentration levels and output characteristic describing whether the levels are acceptable. In some embodiments, the conversion is customized to the patient. This customization may include incorporation of patient-specific variables, such as the patient's weight, to determine whether, for example, the blood analyte concentration level is within an acceptable range. The customization may include learning rules, for example, to determine how the blood analyte concentration level relates to trends in the patient's concentration levels, the variability of the patient's concentration levels, and/or the mean of the patient's concentration levels.

The characteristic may be output by displaying a visual output. For example, the output may comprise a graph. The graph may indicate an acceptable range of blood analyte concentration values (for example by a bar graph or shaded region) and may also indicate the estimated concentration value (for example by an asterisk). The graph may indicate multiple estimated blood analyte concentration values. For example, the graph may comprise a bar graph, wherein each bar indicates an estimated concentration level of a different blood analyte. As another example, the graph may comprise a graph of an estimated blood analyte concentration level as a function of time. In the latter example, methods and/or system described herein may store estimated blood analyte concentration values. The output characteristic may comprise an average of estimated blood analyte concentration values. For example, all estimated concentration values for a given day may be averaged. As another example, estimated concentration values from a given time of day may be averaged across days.

In some embodiments, methods and/or systems may comprise components or may be used to identify potential causes of specific blood analyte concentration values. For example, by calculating the average estimated blood analyte concentration values for a given temperature range, it may be determined that, for example, high concentration values are more common during high temperatures. Additional components may allow for inputs by the user to estimate triggers of specific blood analyte concentration values. For example, the patient may enter food consumed and the output variables may indicate that a specific type of food or characteristic of food is likely to cause high blood analyte concentration values.

It will be understood that in some embodiments, a system and/or device described herein may display additional output characteristic. For example, in an instance in which the device is a watch, the device may also display the time of day. The system and/or device may include an alarm and/or timer component that may be used to indicate when an action is required or suggested by the patient. For example, in an embodiment in which the blood analyte concentration levels are estimated at regular intervals, an alarm may sound regularly throughout the day to alert the patient that the concentration levels should be estimated. In such instances, an alert may be necessary if, for example, the device requires user input, requires a lack of motion by the user, or requires other situational characteristics. The alert may also or instead act as a mechanism to attract the patient's attention to the estimated blood analyte concentration level. As another example, in an embodiment in which the blood analyte concentration levels are continuously estimated or are estimated at regular intervals without requiring the patient's attention and/or input, an alarm may used to alert the user if, for example, the estimated concentration levels are out of the range considered appropriate. The device may comprise an audio transducer, such as a speaker.

Test Data and Adaptation

In order to determine relationships between non-invasively measured data sets and/or between non-invasively measured data sets and a blood analyte concentration level, test or historical data may be used. In these instances, both invasively measured data and non-invasively measured data may be obtained. In preferred embodiments, the data sets are obtained nearly simultaneously. Such test data may be used to adjust or adapt a part of a method and/or system described herein. For example, the test data may be used to set or adapt parameters, equations, and/or other properties related to one or more of pre-processing, nonlinear transformations, source separation processes, and post-processing. In some embodiments, known parameters are used to calibrate a method and/or system described herein. For example, the patient's weight may be used to determine one or more parameters, equations and/or other properties.

In some embodiments, parameters, equations, and/or other properties are determined prior to analysis of the test data. The test data may then be used to verify the accuracy of the pre-determined parameters, equations and/or other properties and/or to alter the pre-determined parameters, equations, and/or other properties. In some embodiments, a plurality of parameters, equations, and/or other properties are identified prior to analysis of the test data. The test data may then be used to determine the preferred parameters, equations, and/or other properties. In some embodiments, parameters, equations, and/or other properties are not identified before analysis of the test data. The parameters, equations, and/or other properties may be identified by, for example, a learning rule.

In some embodiments, test data is used to determine which outputs of a source separation component of a method and/or system described herein is related to a blood analyte concentration level. For example, test data could be used to determine the number of outputs from the source separation component related to the concentration level. These outputs may later be combined. Test data may also be used to determine which of the separated variables is related to the concentration level. For example, test data may reveal that an output with a given variation, strength, auto-correlation and/or spectral property can be identified as related to the concentration level. The test data may be collected for every individual patient, such that, for example, parameters, equations, and/or other properties are optimized for the individual. Alternatively, test data may be collected from one or more individuals to determine appropriate parameters, equations and/or other properties to be used across patients.

In some embodiments, test data may first be collected and then a blood analyte concentration level may subsequently be estimated based on non-invasively measured data. In some embodiments, test data is collected periodically between estimations based only on non-invasively measured data. For example, initial parameters, equations and/or other properties related to one or more of pre-processing, nonlinear transformations, source separation processes, and post-processing may be determined by initial test data or another method. Blood analyte concentration levels may be estimated for a defined interval, such as a week. Test data may then be collected, and the parameters, equations and/or other properties may be adjusted. Alternatively, blood analyte concentration levels may be estimated until there is concern that the estimations are of a specific inaccuracy. For example, if the concentration levels are not varying as much as expected between estimations or are higher or lower than would normally be expected. Test data may then be collected, and the parameters, equations and/or other properties may be adjusted.

Devices

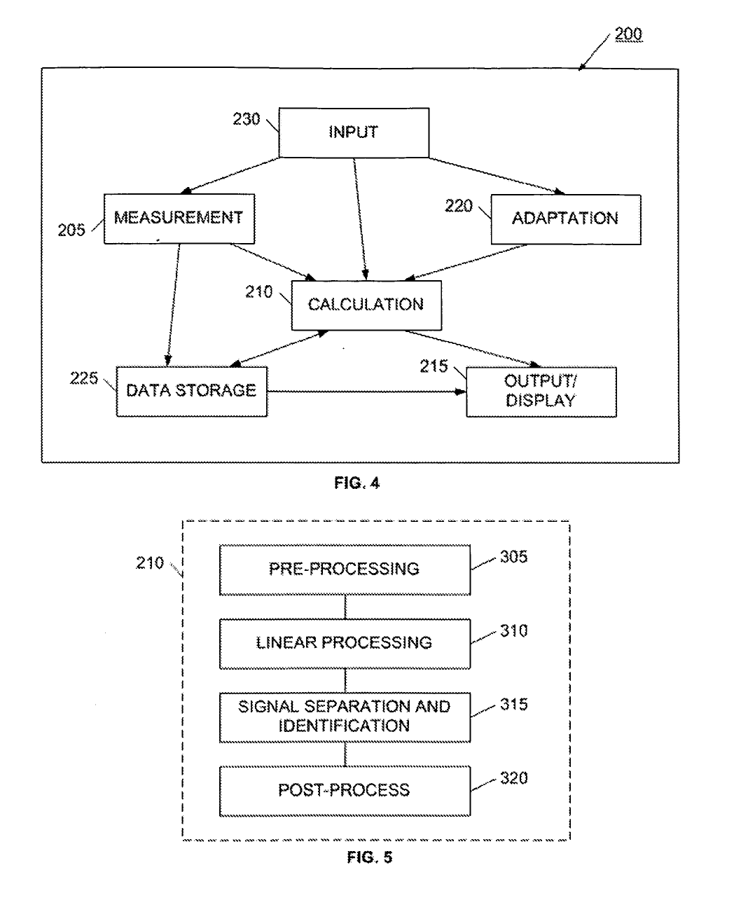

A system described herein may comprise a device, such as device 200 as shown in FIG. 4. The device 200 may comprise a measuring component 205 configured to measure a plurality of physical properties or environmental conditions; a calculation component 210 configured to pre-process at least one data set, nonlinearly transform at least one data set, separate data sets into independent signals, and/or post-process at least one signal; a display component 215 configured to display an output; an adaptation component 220 configured to perform an adaptation according to test data or historical evaluation; a data storage component 225 configured to store data or results; and/or an input component 230 configured to receive user and/or device input. Depending on the embodiment, additional components may be added, others removed, and connections between components may be added and/or removed.

In some embodiments, the device 200 comprises a measurement component 205 configured to measure a plurality of patient physical properties and environmental conditions. The patient physical properties and environmental conditions may comprise any herein. It will be appreciated that some of the measurement or sensor devices may be discreet devices that connect or couple to the device 200. In other cases the measurement or sensor devices may be in a single device 200.

In some embodiments, the measurement component 205 measures an impedance and/or a dielectric property of a patient. The measurement component 205 may comprise one or more electrodes. The one or more electrodes may comprise a capacitive fringing field electrode. The one or more electrodes may comprise two or more electrodes. The two or more electrodes may be spaced apart from each with a separation of, for example, between 200 μm and 4 mm. In other embodiments, the electrodes may be inches or feet in separation. The electrodes may be used to generate electromagnetic fields into the skin and/or various tissue layers underneath the skin of a patient. A plurality of electromagnetic fields may be generated by the electrodes which may achieve different penetrations.

In some embodiments, the measurement component 205 measures a hydration property of a patient. The hydration property may comprise a skin and/or underlying tissue hydration level. The measurement component 205 may comprise a sweat/humidity sensor. The sweat/humidity sensor may comprise an electrode. The electrode may comprise an interdigitated electrode and may utilize a galvanic response based measuring technique.

In some embodiments, the measurement component 205 measures an optical property of a patient. The optical variable may comprise a variable related to the optical properties of the patient's skin. The optical property may be related to the visible spectrum. The measurement component 205 may comprise an optical sensor. The optical sensor may comprise one or more micro-spectrophotometers. The optical sensor may comprise two or more micro-spectrophotometers. The optical sensor may comprise an optical sensor head, which may comprise a fiber-optic transmitter and one, two or more receivers. In some embodiments, the device 200 comprises an input light source. The receivers may be at one, two or more separation distances from the input light source.

In some embodiments, the measurement component 205 measures a pressure property of a patient. The pressure property may comprise a variable indicating the pressure of the device on the patient's skin. The measurement component 205 may comprise a piezoelectric element. The piezoelectric element may be an integrated piezoelectric sensor. In some embodiments, the measurement component 205 measures a movement property of a patient. The movement property may comprise a variable indicating the movement of the device 200. The movement of the device 200 may be absolute or relative to, for example, a movement of the patient. The measurement component 205 may comprise an accelerometer. In some embodiments, the measurement component 205 measures a weather-related condition. The weather-related condition may comprise a temperature, a pressure variable and/or a humidity variable. The measurement component 205 may comprise a thermometer, barometer, psychrometer and/or hygrometer. In some embodiments, the measurement component 205 measures a capacitance property of a patient and/or a current property of a patient. In some embodiments, the measurement component 205 measures one or more of body temperature, skin temperature, blood flow, blood pressure, an ECG variable, an EEG variable, and an oxygen saturation variable. The measurement component 205 may include a component to measure any of these properties or conditions.

In some embodiments, the measurement component 205 measures both an analyte-sensitive physical property and an analyte-insensitive physical property. In other embodiments, the measurement component 205 measures either an analyte-sensitive physical property or an analyte-insensitive physical property. The device 200 may receive one or more analyte-sensitive data sets and/or one or more analyte-insensitive data sets via the input component 230. In some embodiments, the measurement component 205 measures an analyte-sensitive physical property and the input component 230 receives an analyte-insensitive physical property or condition as an input.

In some embodiments, the device 200 includes a calculation component 210. The calculation component 210 may comprise one or more of a pre-processing component 305, a nonlinear calculation component 310, a source separation component 315, and a post-processing component 320, as shown in FIG. 5. Depending on the embodiment, additional components may be added, others removed, and connections between components may be added and/or removed. For example, the pre-processing component 305 and/or the post-processing component 320 may be removed from the calculation component 210.

In some embodiments, the calculation component 210 includes a pre-processing component 305. The pre-processing component 305 may pre-process one or more measured data sets which may be provided by the measurement component 205 of the device 200 and/or one or more input data which may be provided by the input component 230 of the device 200. The pre-processing may include a variety of processes, such as a normalization process. Pre-processing may also include the combining data sets from two or more measurements. For example, two or more impedance readings may be combined into a single data set.

In some embodiments, the calculation component 210 includes a nonlinear calculation component 310 configured to nonlinearly transform at least one data set. The at least one data set may comprise data sets measured by the measurement component 205 of the device 200, data input via the input component 230 of the device 200, and/or a data processed by the pre-processing component 305 of the calculation component 210. The at least one data set may comprise an analyte- sensitive data set and/or an analyte-insensitive data set. The at least one data set may comprise a pre-processed data set and/or a data set that has not been pre-processed. The nonlinear calculation component 310 may include constraints that may comprise a priori constraints and/or derived constraints. The nonlinear calculation component 310 may include one or more learning rules.

In some embodiments, the calculation component 210 includes a linear calculation component. The linear calculation component may comprise a source separation component. The calculation component 310 may include a source separation component 315. The source separation component 315 may comprise a blind source separation module configured to separate at least two signals. The blind source separation module may comprise, for example, an ICA and/or an IVA module. The linear calculation component and/or the source separation component 315 may be configured to identify one or more signals related to a blood analyte concentration level. The linear calculation component and/or the source separation component 315 may receive as inputs one or more of measured data sets provided by the measurement component 205 of the device 200, input data provided by the input component 230 of the device 200, pre-processed data sets provided by the pre-processing component 305 of the calculation component 210, and nonlinearly transformed data sets provided by the nonlinear calculation component 310 of the calculation component 210.

In some embodiments, the calculation component 210 includes a post-processing component 320. The post-processing component 320 may be configured to scale and/or impose an offset to one or more signals from the separation process 315. The post-processing component 320 may be configured to combine signals and/or to identify a desired signal. The post-processing component 320 may be configured to calculate the confidence and/or error of a blood analyte concentration level estimate. The post-processing component 320 may be configured to convert a blood analyte concentration level estimate into an output form. The post-processing component 320 may act on one or more of measured data sets provided by the measurement component 205 of the device 200, input data provided by the input component 230 of the device 200, pre-processed data sets provided by the pre-processing component 305 of the calculation component 210, nonlinearly transformed data sets provided by the nonlinear calculation component 310 of the calculation component 210, and separated signals provided by the source separation component 315 of the calculation component 210. The post process 320 may also provide scaling, filtering, or other analytical functions.

In some embodiments, the device 200 may comprise multiple components on, near, adjacent or far from the other components. For example, the output component may be separate from the sensors. The different components can be connected wired or wireless.

In some embodiments, the device 200 comprises an output component that may output one or more output signals, results, or data. The output may be displayed on a display component 215. The display component 215 may display one or more of numbers, text, instructions, graphs, tables, charts and pictures. The display component 215 may display information related to a blood analyte concentration level and/or information unrelated to a blood analyte concentration level (e.g., the time of day). The display component 215 may display information provided by the calculation component 210. The display component 215 may display a history related to blood analyte concentration levels. The display component 215 may display estimated blood analyte concentration levels as a function of time.

In some embodiments, the device 200 comprises an adaptation component 220. The adaptation component 220 may comprise a test data component. The test data component may, for example, compare invasively measured or otherwise known blood analyte concentration levels with blood analyte concentration levels estimated from non-invasively measured data sets. The adaptation component 220 may determine parameters, equations and/or other properties related to other components (e.g., the calculation component 210) based on test data and/or information, for example, about the patient, such as the patient's weight. In some embodiments, the adaptation component 220 is only used during initial setup of the device. In some embodiments, the adaptation component 220 is used subsequent to the initial setup. The adaptation component 220 may be used on regular or irregular intervals. The adaptation component 220 may comprise, for example, learning rules.

In some embodiments, the device 200 comprises a data storage component 225. The data storage component 225 may store, for example, estimated blood analyte concentration levels which may be provided by the calculation component 210. The data storage component 225 may store one or more measured data sets, results, or interim values. The data storage component 225 may store one or more output signals or results. In some embodiments, the data storage component 225 may provide stored data to the calculation component 210. The calculation component 210 may, for example, use the stored data to calculate average estimated blood analyte concentration levels or trends in the concentration levels. In some embodiments, the data storage component 225 may provide stored data to the display component 215. The display component 215 may, for example, use the stored data to show trends in the concentration levels as a function of time.

In some embodiments, the device 200 comprises an input component 230. The input component 230 may include a mouse, a keyboard, and/or one or more buttons. The input component 230 may include a responsive screen, such as a touch-sensitive screen. The input component 230 may include an input/output port or an electrical connection. For example, the input component 230 may comprise a USB port. The input component 230 may be configured to receive variables, such as variables measured by another device. The input component 230 may be configured to receive instruction or information from the user, such as a list of food eaten or the time of one or more previous treatments (e.g., insulin injections). The input component 230 may be configured to receive inputs related to the blood analyte concentration level estimations. For example, the inputs may be used to change a parameter and/or equation of a component of the calculation component 210. As another example, inputs may be used to identify concentration level estimates to be averaged (e.g., an input could indicate that all concentration levels within each day be averaged). Inputs may also indicate that it is time for an estimation to be made and/or time for the measurement component 205 to measure one or more physical properties or conditions. Inputs may be used to provide test data to the calibration component 220 comprising, for example, invasively measured variables to the device or may indicate that it is time for a calibration to be performed by the calibration component 220. Inputs may control display settings of the device 200. Inputs may control data stored in the memory of the device 200. In some embodiments, the device 200 comprises a computer.

In some embodiments, the device 200 can be worn by a patient. The device 200 may comprise, for example, a watch. The device 200 may comprise a band, such as a wrist band or an ankle band. The device 200 may comprise a glove. The device 200 may comprise a patch. In some embodiments, the device 200 continuously estimates a blood analyte concentration level. In some embodiments, the device 200 regularly estimates a blood analyte concentration level. In some embodiments, the device 200 estimates a blood analyte concentration level after receiving a specific user input.

Computer Implementation

In some embodiments, a method described herein comprises a computer-implemented method. In some embodiments, a system and/or device described herein comprises a computer. In some embodiments, a calculation component of a system and/or device described herein comprises a computer. The computer may comprise a digital signal processor (DSP) or a central processing unit (CPU), one or more peripherals (e.g., RAM, ROM, PROM, or EPROM), and a program to be executed by the DSP or CPU. The computer may comprise an input device, which may be configured to receive data from another device and/or to receive input data from a user. The computer may comprise a user interface for receiving or displaying data and/or information. The computer may comprise an output device, which may be configured to display data and/or information. The program may comprise computer-readable medium comprising instructions for performing a method disclosed herein.

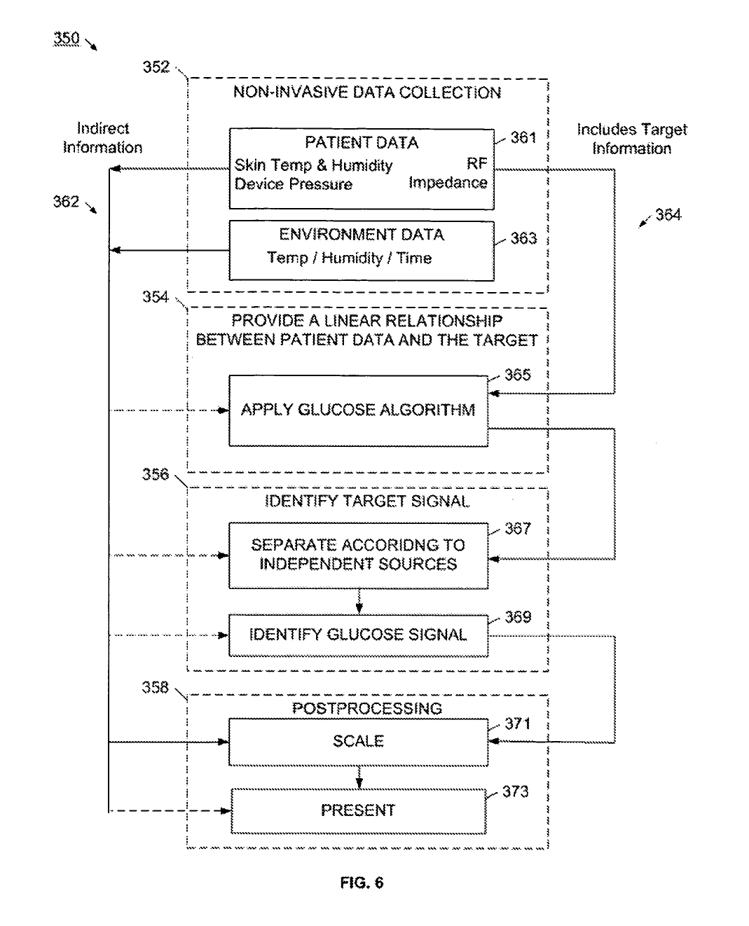

Referring now to FIG. 6, a glucose characterization method 350 is illustrated. Generally, glucose characterization process 350 has four steps: first 352, data is collected from the patient and the environment using non-invasive techniques; second 354, a generally linear relationship is provided between the collected data and the glucose level; third 356, a glucose signal is identified from the data set that is indicative of the glucose level; and fourth 58, the glucose signal is scaled and processed for presentation. These steps enable glucose characterization process 350 to present a highly accurate glucose level, even under changing patient or environmental conditions. In this way, process 50 may be implemented in a wider range of applications and environments, and may be used with greater confidence and less pain than previous devices or processes.

In step 352, noninvasive data is collected from the patient 361 or from the environment 363. Typically, data is collected from the patient using noninvasive skin-surface sensors. These sensors may be used to measure electrical, optical, temperature, or humidity characteristics, for example. Some of these characteristics may measure surface characteristics, while others may indicate characteristics of underlying tissue or fluids. Some of the sensors may be configured to measure an existing property, such as temperature, while other sensors may actively provide a stimulation. For example, some sensors may provide an RF frequency signal for measuring an RF impedance, while other sensors may provide a light signal for measuring an optical property. It will be understood that a wide range of noninvasive sensors may be used. At least some of the data collected from the patient 361 has information that has a known direct relationship with the target physiological parameter as shown by arrow 364. This means, for example, that a change in the glucose level causes a change in that set of data. Other data from the patient and from the environment may have an indirect relationship as shown by arrow 362.

In a specific example of process 350, RF Impedance data is collected from the patient using a non-invasive sensor. The RF data has a known direct relationship with the glucose level. Other patient data, such as the skin temperature and skin humidity is measured, as well as the pressure between the sensor and the skin. This latter data does not directly indicate any glucose level, but is used for adjusting other aspects of process 350 that provide scaling, calibration, or filtering, for example.

The RF data 364, which has a known non-linear relationship with the glucose level, is passed through an algorithmic, table, modeling, or other scaling process 365 to adjust the RF data for a more linear relationship. This linearized data is then passed to identification step 356. The linearization process 354 may be determined according to historical data, published data, or learned and adapted over time. In block 356, the linearized data is first separated into independent sources as shown in block 367, and then the signal associated with the glucose level is identified in block 369. Typically, the separation process will be a blind signal source process, for example, an independent component analysis, or may have another signal separation process applied. With the proper signal identified, the glucose signal is scaled 371, typically using the indirect information 362. The scaled glucose level is then presented as shown in block 373.

Patients

A method and/or system described herein may be used to determine a desired physiological parameter such as blood analyte concentration level of a patient. A system described herein may be provided to a patient. A patient may provide a third party, such as a physician, with a plurality of data sets. The physician may then use a method and/or system described herein to estimate a blood analyte concentration level of the patient. For example, the third party may apply a nonlinear transformation to at least one of the plurality of variables and then employ a source separation process and a post-processing technique to estimate the blood analyte concentration level. In this way, the patient may wear a smaller device constructed to measure and collect data sets of measured physical properties or environmental conditions. When analysis is desired, the data sets are loaded onto a processing device that applies the previously discussed processes. In some embodiments, a kit comprising instructions described herein of estimating a blood analyte concentration level is provided. Accordingly, the particular modules of device 200 may be found on multiple discrete devices.

In some embodiments, a method and/or system described herein estimates a pre-determined blood analyte concentration level, such as glucose. In some embodiments, a method and/or system described herein estimates a plurality of pre-determined blood analyte concentration levels, such as glucose and cholesterol. In some embodiments, a method and/or system described herein screens for potential conditions by estimating a plurality of blood analyte concentration levels. In some embodiments, the patient is healthy.

In some embodiments, the patient suffers from a known condition (e.g., a blood-glucose condition), while in other embodiments, the patient is at risk of suffering from the condition. The patient may be at risk of suffering from the condition due to, for example, a family history, a disease history, a glucose test history (e.g., impaired fasting glucose or impaired glucose tolerance), an insulin condition (e.g., insulin resistance), a weight condition (e.g., obesity), high blood pressure, a cholesterol condition (e.g., HDL cholesterol less than 35 mg/dL or triglyceride levels greater than 250 mg/dL), a metabolic disorder (e.g., polycystic ovary syndrome), being of a specific ethnicity, and/or a blood vessel condition. The condition may be related to a blood analyte concentration level. The condition may be related to an abnormal blood analyte concentration level. The condition may be related to glucose levels. The condition may be insulin resistance. The condition may be diabetes. The condition may be impaired glucose homeostasis and/or impaired glucose tolerance. In other embodiments, the patient is unaware of a known condition, and the invention method and device is utilized to detect a particular condition.

The diabetes may be Type 1 or Type 2 diabetes. Type 1 (or insulin-dependent diabetes mellitus or juvenile-onset diabetes), develops when the body's immune system destroys pancreatic cells that make the hormone insulin, which regulates blood glucose levels. Type 1 diabetes usually occurs in children and young adults, although disease onset can occur at any age. Type 1 diabetes accounts for about 5 to 10 percent of all diagnosed cases of diabetes. Risk factors for Type 1 diabetes include autoimmune, genetic, and environmental factors. Type 2 (or Type II) diabetes (non-insulin-dependent diabetes mellitus (NIDDM) or adult-onset diabetes), is a metabolic disorder involving dysregulation of glucose metabolism and insulin resistance, which can result in long-term complications involving the eyes, kidneys, nerves, and blood vessels. Type 2 diabetes results from the body's inability to make either sufficient insulin (abnormal insulin secretion) or its inability to effectively use insulin (resistance to insulin action in target organs and tissues). This disease usually begins as insulin resistance, a disorder in which the cells do not use insulin properly, and as the need for insulin rises, the pancreas gradually loses its ability to produce insulin. Patients suffering from Type 2 diabetes have a relative insulin deficiency. That is, in these patients, plasma insulin levels are normal to high in absolute terms, although they are lower than predicted for the level of plasma glucose that is present. Type 2 diabetes is the most common form of the disease accounting for 90-95% of diabetes.

The patient may be of any age. The patient may be under the age of 18. The patient may be over the age of 65. The patient may be experiencing or may have experienced one or more of persistently elevated plasma glucose concentration (hyperglycemia); polyuria; polydipsia; polyphagia; chronic microvascular complications such as retinopathy, nephropathy and neuropathy; and macrovascular complications such as hyperlipidemia and hypertension. The patient may be experiencing or may have experienced blindness, end-stage renal disease, limb amputation and myocardial infarction. Any of these symptoms may be a symptom of diabetes.

The patient may be suffering from or at risk of suffering from gestational diabetes. Gestational diabetes refers to a form of glucose intolerance that is diagnosed in pregnant women. The patient may be pregnant. A percentage (5-10 percent) of women with gestational diabetes have Type 2 diabetes after pregnancy. Women who have had gestational diabetes also have a 20-50 percent chance of developing diabetes in the next 5-10 years. The patient may have recently or previously been pregnant. The patient may be receiving a treatment to treat a condition. The condition may be any condition described herein or any other condition. The treatment may comprise an insulin treatment. The treatment may be administered routinely or on an as-needed basis. The routinely administered treatment may be administered daily. A method and/or system descried herein may be used to determine when a treatment should be administered and/or provided to the patient.

Kits

In some embodiments, a kit comprises a system and/or device described herein. In some embodiments, a kit comprises a set of instructions providing a method described herein. In some embodiments, a kit comprises a set of instructions related to use of a system and/or device described herein. In some embodiments, a kit comprises a set of instructions related to the interpretation of output from a system, device and/or method described herein. Instructions may indicate how frequently a system, device, and/or method described herein should be used. Instructions may indicate appropriate blood analyte concentration levels. The appropriate blood analyte concentration levels may be determined by standard healthcare knowledge. Instructions may suggest actions when high and/or low blood analyte concentration level estimates are provided by a system, device and/or method described herein. Instructions may indicate proper usage of a system and/or device described herein. For example, the instructions may indicate details related to preferred and/or necessary skin contact with the device. Instructions may suggest when the system and/or device should be calibrated.

Uses

In some embodiments, a device and/or a method described herein may be used as part of a treatment for a condition (e.g., diabetes). For example, the device and/or method could provide data useful in determining a dosage of a drug that should be administered or a time when a drug should be administered. In some embodiments, a device and/or a method described herein may used as part of a dietary regimen and/or a weight loss program. The dietary regimen and/or the weight loss program may be related to a health-related condition. For example, the device and/or method may be used by a patient with high cholesterol. The device and/or method may then indicate when, for example, it is recommended that a patient intake or not intake a particular type of food. In some embodiments, a device and/or a method described herein may be used as an information source by a medical professional. For example, the device and/or method may provide a means by which a doctor can monitor a patient's glucose levels between visits. In such embodiments, estimated glucose levels may, for example, be sent to the medical professional via a network connection. As another example, estimated blood analyte concentration levels may be analyzed across patients. Such analysis may suggest particular causes of specific levels, pre-dispositions to specific levels, effective treatments, and/or trends in patients' health.

### EXAMPLE 1

Data Measurement. Various sensors to monitor different physiological properties were used to acquire information about patients by generating one or more data sets. Both healthy and patients with diabetes were tested, although other patients could be easily tested. Different information was acquired using multiple sensors on the skin, particularly the forearm. Although the forearm was tested, the sensors could be placed on different parts of the body or on one discrete point. Eventually, as non-contact sensors are developed, they can be placed adjacent to the skin, although direct contact with the skin is preferred. The measurement data included RF (radio frequency) impedance in conjunction with the temperature (skin and device), humidity and pressure between skin and device. The RF impedance is a primary signal of interest, but environmental conditions such as temperature and humidity also used to calibrate personal and environmental changes.

Information that was acquired at the point of contact(s) was the dielectric properties of the skin and the underlying tissue, pressure, temperature, moisture and blood perfusion, although other physiological measurements have been taken as well including pulse and other cardiovascular information, and the like. Impedance information was acquired using a depth selective electrical impedance spectrometer (e.g., dielectric spectroscopy based differential sensor, bioimpedence analyzers such as Quantum X™ (RJL Systems, Inc., Clinton Twp., Mich.), HYDRA ECF/ICF 4200 Bio-Impedance Spectrum Analyzer (Xitron Technologies, Inc., San Diego, Calif.), SciBase II spectrometer (SciBase AB, Huddinge, Sweden), Solianis spectrometer (Solianis AG, Zurich Switzerland), Fusion XS™ Spectrometer (Biopeak Corp, Ottawa, Canada), and the like). Other physiological signals were also acquired, for example, temperature from infrared temperature sensor, piezoelectric sensor to detect pressure and an optical sensor to measure perfusion or capillary blood flow (e.g., laser Doppler flowmetry—Periflux™ Pf2 (Perimed AB, Stockholm, Sweden).

Linear Processing. Empirically it is found that the RF measurement data is in nonlinear relationship with the glucose level, and the nonlinearity needs to be reduced before linear prediction stage. This linear processing step nonlinearly transforms or adapts the RF data such that the transformed data set has a more linear relationship with the glucose level. For the purpose of providing a more linear relationship between the measured data and the glucose level, we use a 1D mapping function f(.), to compute preprocessed signal Y.

yi=fi(xi) (Eq. 2)

Where X=[x1 , , , xn], Y=[y1 , , , yn]

Specifically in this example, we use a function that scales the signal nonlinearly in order to adjust the data set so that it has a more linear relationship with glucose levels. It will be understood that after the linearization process, the resulting relationship is not fully linear, but is a more linear relationship than prior to the transformation. As more data is collected and analyzed, other functions, tables, or algorithms may be used to provide enhanced linearity. Different mapping functions can be designed for each of data dimensions. Examples of such nonlinear scaling functions are:
f(x)=ax̂b+c;
f(x)=cosh(x);
f(x)=1/(1+exp(−x)); and
f(x)=log (x).

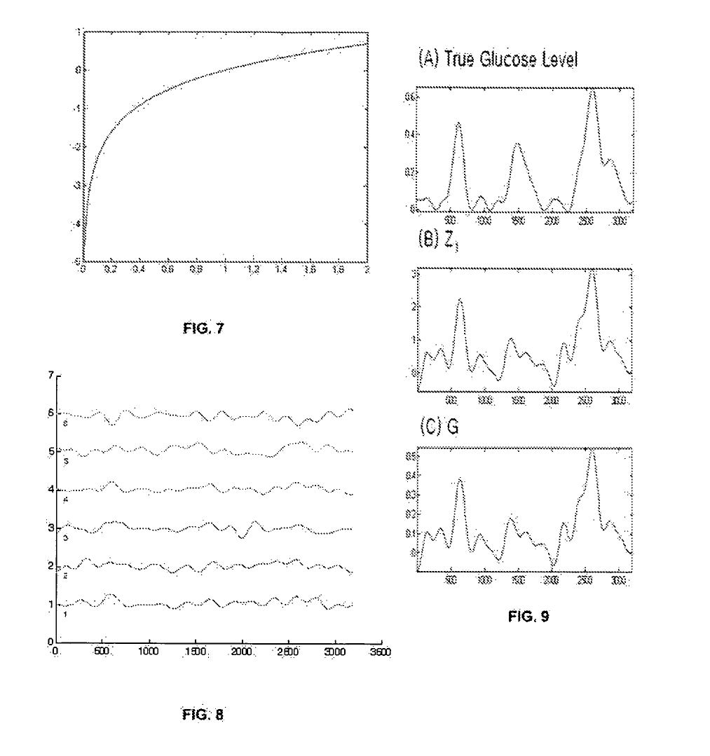

Another non-linear mapping function is illustrated in FIG. 7. It will be appreciated that look-up tables, models, or other transformation processes may be used.

Independent Source Separation. From the transformed test data set, we can compute a linear mapping z which should be highly correlated with the glucose level and be robust to personal and environmental changes. ICA (Independent Component Analysis) is an algorithm that identifies linear subspace of independent components from a set of input signals. When applied to the transformed signal, ICA finds number of subspaces of which signals are independent of each other. ICA is able to identify linear components which are independent of each other. The pre-processed RF impedance signal is linearly correlated with the glucose level, but is highly contaminated with the other noisy factors. Since ICA can extract original signal from multi-dimensional observation signals mixed with high noise, we can find cleaner signals which shows higher correlation with the glucose level. The component 1(Z1) of the ICA source signals shows high correlation with the original glucose levels. FIG. 9(A) and (B) show the comparison of ICA source signal Z1 with the true glucose level measured by invasive method. The ICA source Z1 shows high correlation coefficient (0.87). While the input data X shows minimal correlation in FIG. 8.

Post-Processing. After the glucose predictor is computed, we compensate the scale and offset of the value to be matched with standard glucose level by equation (3).

G=cZ1+offset (Eq. 3)

The calibration constant c and offset can be estimated using small number of actual glucose level measurements. FIG. 9(C) shows the final estimated glucose level G which shows high correlation with the true glucose level of FIG. 6(A).

### EXAMPLE 2

In a second example, multiple sets of RF data were collected. Several sets of RF data were collected, with each set representing impedance at a different skin depth. In another example, each set represents RF impedance measured at a different frequency, or using a different signal shape, under different positions or placements of the sensors, or over a period of time. Several datapoints of blood analyte information can be analyzed over a period of time because gradual changes typically occur over several minutes. In this way, example 2 uses multiple sets of the same type of data, with each data set having a known direct relationship with glucose or another target physiological parameter. By using multiple sets of the same type of data, reliance on other indirect data may be eliminated or reduced.

### EXAMPLE 3

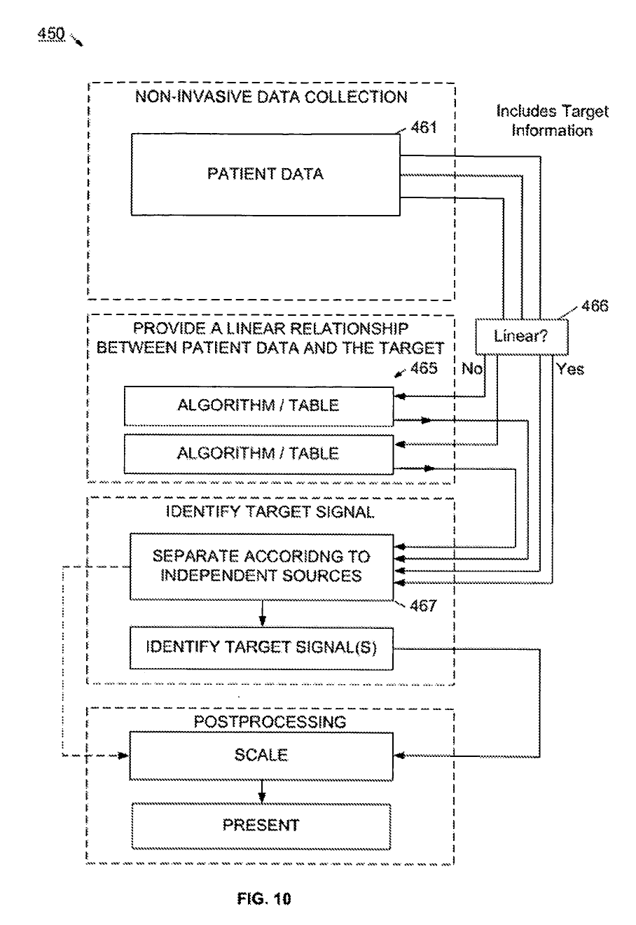

In a third example, multiple sets of direct data are collected. For example, a set of RF data may be collected, and a set of infrared data may be collected. In this way, example 3 uses multiple sets of different direct data, with each data set having a known direct relationship with glucose or another target physiological parameter. By using multiple sets of direct data, reliance on other indirect data may be eliminated or reduced. It will be understood that many different types of direct data sets may be substituted or used. For example, direct data may include RF impedance data, near infrared data, far infrared data, polarization data, or florescence data, for example. By using multiple direct data sets, increased accuracy and reliability may be obtained, while reducing reliance on other indirect data measurements. FIG. 10 generally shows such a process. Process 450 is similar to processes 50 and 350 previously described, so will not be discussed in detail. In characterization process 450, only data having a direct relationship with the physiological parameter is collected, as shown in block 461. However, multiple sets are collected. In one example, the multiple sets represent different collections of the same type of data (eg all RF impedance data, but at different skin depths). In another example, the multiple sets each represent different types of data (eg, one set of RF impedance data and one set of infrared data). In yet another example, some data sets may have different collections of the same type of date, and other data sets may have different types of data. It will be understood that a wide range of direct data types and collection specifics are possible. In block 466, it is determined if each data set has a linear relationship with the target physiological parameter. If so, the data is passed to the identification process, and if not, each non-linear data set is linearized using one or more algorithms/tables/models 465. The generally linear data is received into the separation process 467. It will be understood that a single separation process may be used, where each of the linear data sets becomes an input signal to a single process, or that multiple separation processes may be used. The identified signal or signals are scaled and presented.

While the above detailed description has shown, described, and pointed out novel features of the invention as applied to various embodiments, it will be understood that various omissions, substitutions, and changes in the form and details of the device or process illustrated may be made by those skilled in the art without departing from the spirit of the invention. The scope of the invention is indicated by the appended claims rather than by the foregoing description. All changes which come within the meaning and range of equivalency of the claims are to be embraced within their scope.

1-76. (canceled)

77. A method selected from the group consisting of:
a. estimating a concentration level of a blood analyte, optionally glucose, comprising:
(i) non-invasively measuring a plurality of variables in a patient to obtain a set of input data, optionally by emitting at least one pair of wavelengths (optionally within the range of about 600 to about 1 millimeter) in the from an energy source towards a first selected area of the patient and detecting energy emerging from a second selected area of the patient wherein at least one first variable of the plurality of variables depends on the patient's blood analyte concentration level and optionally comprises a variable selected from electrical impedance variable (optionally an impedance spectroscopy variable), a capacitance variable (optionally a skin capacitance variable), and a current variable, wherein at least one second variable of the plurality of variables does not depends on the patient's blood analyte concentration level, and wherein optionally the at least one first variable depends on the at least one second variable;
(ii) nonlinearly filtering at least part of the set of input data to obtain a set of filtered data, wherein optionally the nonlinearly filtering comprises an at least partially adaptive component; and
(iii) applying a source separation method to the set of filtered data to obtain a set of output data, wherein optionally the source separation method is at least partially adaptive;
b. estimating a blood-analyte concentration level in a patient, wherein the blood-analyte optionally is glucose comprising:
(i) receiving a first set of input variables, wherein the first set of input variables do not comprise any invasively-measured variables, wherein at least one first variable of the first set of input variables is influenced by the patient's blood analyte concentration level, and wherein at least one second variable of the first set of input variables is not influenced by the patient's blood analyte concentration level;
(ii) pre-processing at least one of the first set of input variables to produce a second set of variables, wherein the pre-processing optionally is at least partially adaptive and optionally comprises nonlinearly transforming at least one of the first set of input variables; and
(iii) applying a linear separation method to the second set of variables produce a third set of variables, wherein the linear separation method optionally is at least partially adaptive and optionally comprises a blind source separation method (optionally at least one of an Independent Component Analysis (ICA) and an Independent Vector Analysis (IVA) method); and
c. characterizing a target physiological parameter, comprising:
(i) collecting a first data set of data from a patient, the first data set having a direct relationship with the target physiological parameter, wherein collecting the first data set optionally further comprises using an optical, electrical, RF, infrared sensor, or impedance sensor;
(ii) collecting a second data set, wherein the second data set optionally is a set of data having a direct or indirect relationship with the target physiological parameter, wherein the second data set optionally is or is not indicative of a physiological parameter and/or an environmental condition, and wherein the second data set optionally is from a patient;
(iii) processing the first data to generate a processed first data set that has a generally linear relationship with the target physiological parameter, wherein the processing step optionally comprises at least one of determining that the first data set has a generally linear or nonlinear relationship with the processed first data set and/or applying an algorithm or table to the first data set to generate the processed first data set;
(iv) separating the processed first data set into independent signals, wherein the separation process optionally is a blind signal separation process or an independent component analysis process, and wherein the separation step optionally is adapted according to the second data set;
(v) identifying a parameter signal having the target physiological parameter as its source, wherein the identification step optionally is adapted according to the second data set;
(vi) scaling the parameter signal according to the second data set, wherein the scaling step optionally is adapted according to the second data set; and
(vii) presenting the scaled parameter, wherein the presenting step optionally comprises visually displaying, audibly projecting, setting an alarm, sounding an alarm, communicating a message, or activating another device.

78. A method according to claim 77(a) wherein the plurality of variables comprises at least one variable selected from the group consisting of skin temperature, body temperature, air temperature, skin moisture, blood flow, blood pressure, a hydration variable, an ECG variable, an EEG variable, a skin-device pressure variable, device movement, atmospheric pressure, an oxygen saturation variable, and humidity, and optionally the time of day.

79. A method according to claim 77(a) further comprising invasively measuring a variable dependent on the blood analyte, and optionally even further comprising comparing the invasively-measured variable to at least one or more of the plurality of variables, at least one variable of the set of filtered data, and at least one variable of the set of output data.

80. A method according to claim 77 wherein the source separation method comprises at least one of an Independent Component Analysis (ICA) and an Independent Vector Analysis (IVA) method.

81. A method according to claim 77(b) further comprising at least one the following:
a. post-processing at least one of the third set of variables;
b. determining the nonlinear transform by using test data comprising both non-invasively measured variables and invasively measured variables;
c. determining the nonlinear transform by using a neural network to relate test data comprising non-invasively measured variables to test data comprising invasively measured variables;
d. determining parameters of the linear separation method by using test data comprising both non-invasively measured variables and invasively measured variables; and/or
e. determining parameters of the linear separation by using a neural network to relate test data comprising non-invasively measured variables to test data comprising invasively measured variables.

82. A method according to claim 77(c) wherein the first data set has a generally linear relationship with the target physiological parameter so that the processing step does not change data values in the first data set.

83. A method according to claim 77(c) wherein the target physiological parameter is selected from the group consisting of blood analyte, cancer detection, heart condition, hydration, fat composition, tissue characterization, blood flow/pressure, electrolyte, fat, hemoglobin, lactic acid, oxygen saturation, and respiration.

84. A method according to claim 77 that is a computer-implemented method.

85. A non-invasive blood-analyte-monitoring apparatus, comprising:
a. an analyte-sensitive measuring component configured to measure an analyte-sensitive variable related to a concentration level of a blood analyte, optionally glucose, in a patient, wherein the analyte-sensitive variable optionally comprises a variable selected from the group consisting of an impedance variable (optionally an electrical impedance variable), a capacitance variable, and a current variable;
b. an analyte-insensitive measuring component configured to measure an analyte-insensitive variable not related to the concentration level of the blood analyte in the patient, wherein the analyte-insensitive variable optionally comprises a variable selected from skin temperature, body temperature, air temperature, skin moisture, a hydration variable, a skin-device pressure variable, atmospheric pressure, device movement, and humidity;
c. an analyte calculation component comprising a nonlinear calculation component that is configured to nonlinearly filter at least one variable, wherein the analyte calculation component is configured to receive the analyte-sensitive and analyte-insensitive variables as inputs and calculate the patient's estimated blood analyte concentration level, wherein the analyte calculation component optionally is at least partially adaptive and/or comprises a blind source separation module configured to separate at least two signals (wherein the blind source separation module optionally comprises at least one of an Independent Component Analysis (ICA) module and an Independent Vector Analysis (IVA) module), wherein the nonlinearly filtering optionally comprises taking the logarithm of the at least one variable.

86. An apparatus according to claim 85 wherein the analyte-sensitive measuring component comprises at least one electrode.

87. An apparatus according to claim 85 further comprising at least one of the following:
a. a stimulus-delivering component, wherein the stimulus-delivering component optionally comprises at least one electrode;
b. a temperature-measuring component;
c. a pressure-measuring component;
d. an optical sensor;
e. a display component, wherein the display component optionally is configured to display (i) the patient's estimated blood analyte concentration level and/or (ii) the patient's estimated blood analyte concentration level as a function of time; and/or
f. a data storage component, wherein the data storage component optionally stores estimated blood analyte concentration level data.

88. An apparatus according to claim 85 that comprises a watch.

89. A glucose monitor, comprising:
a. a housing;
b. a first sensor that is a non-invasive sensor configured to collect RF impedance data, wherein the first sensor optionally is disposed in the housing;
c. a second sensor configured to collect other patient data, wherein the second sensor optionally is disposed in the housing;
d. a display in the housing for presenting a measured glucose level; and
e. a processor in the housing for operating the steps of:
(i) receiving the set of RF impedance data;
(ii) linearizing the RF impedance data to glucose;
(iii) separating the linearized data using a blind signal source algorithm;
(iv) identifying a glucose signal;
(v) scaling the glucose signal according to the other patient data; and
(v) presenting the scaled glucose signal as the measured glucose level.

90. A glucose monitor according to claim 89 wherein the non-invasive sensor and the second sensor are each disposed in the housing.

91. A glucose monitor according to claim 89 wherein the other patient data is selected from the group consisting of skin temperature, skin humidity, pressure between the first sensor and the skin, and ambient temperature.

92. A glucose monitor according to claim 89 wherein the processor further uses the other patient data to filter noise from the RF impedance data.
